SEQUENCE LISTING (3 sequences)

SEQ ID NO: 1 - DNA, 5696 bp, Homo sapiens
aatcgcgcacaatgagacagcgctgagcgccggaagtggggccgaaggaaaacacggagagggagcccggccgggacaggaagaggctggggaccgcggcgaaggtggtgagtgctcttgggcgccttctcccaacgtccctgccagactcgcctccgggctgattctccagttggtttcctggactccagagtagctgtccggcctggccccggaggtgcaaagtaagaaaattgaagtcaaagaccatgggagatacagcaaaaccttatttcgtgaagcgcactaaagaccgggggactatggatgatgatgacttcagaaggggtcacccccaacaagattatttaataatagatgaccatgctaaaggccatggcagtaaaatggaaaagggccttcaaaaaaagaagataacaccagggaactatgggaatacccccagaaaaggaccatgtgctgtttccagcaatccatatgcatttaaaaacccaatctacagtcaacccgcttggatgaatgacagccacaaagatcagagtaagagatggctgtctgatgaacatactggtaattcagacaactggagagaattcaaacctggacctagaattcctgttataaaccgacaaagaaaagactcctttcaagaaaatgaagatggttataggtggcaagacacaagaggctgcagaactgtaagacgactgtttcataaagacctaacaagcctagaaaccacgtcagaaatggaagcaggaagtcctgaaaacaagaagcagaggtccagacctaggaagccacggaagactagaaatgaggaaaatgagcaggatggagacttggaaggccctgtgatcgatgagtctgtactttcaacgaaggagctgctaggcttacagcaggctgaggagagactgaagagagactgcattgacaggctaaaaaggcgaccacgaaactaccctacagcaaagtacacctgcagactctgtgatgttttaattgaatccattgcatttgcccataagcatatcaaggaaaagaggcacaagaaaaacattaaggagaagcaagaggaagagttgctcactacgttacccccaccaacaccctcccagataaatgcagttggcattgccattgacaaagtggtacaggaatttggcttacacaatgagaacttggaacagaggctggaaattaaacgtatcatggaaaatgtgttccaacacaagttaccagattgttccctaagattatatgggtcatcctgtagcagattgggtttcaaaaattcggatgtaaacattgacatccagtttccagccattatgtctcagccagatgtcctcttacttgttcaagaatgtttaaagaacagtgactcctttattgatgttgatgcagacttccatgctagggtgccagtggtggtgtgcagagaaaagcaaagtggtcttctgtgtaaagtgagcgcaggaaatgaaaatgcttgtctgacaacaaagcatttaactgcccttggaaaactagaaccaaagctggttcctttggtgattgcatttaggtactgggcaaagctttgcagtatagatcgccctgaagaaggaggtctgccaccttatgtgtttgccctgatggccattttctttcttcagcagaggaaagaaccccttttgcctgtatatctaggatcatggattgaaggattctcattaagcaaactagggaatttcaaccttcaagacattgaaaaagatgttgtgatctgggaacatactgacagtgctgcaggggacacaggcataacaaaagaagaggcaccaagagaaacgccgattaaaaggggacaggtgtcattaatattggatgtgaaacaccagccttcagtaccagttgggcagctctgggtggaattgctgcggttctatgctttagaatttaatttggctgatttagtgataagtattcgtgtcaaagaattggtatctcgggaattgaaggattggcccaaaaagcgcattgccattgaagatccctactctgttaaaagaaatgtggcaagaaccctaaatagtcaacctgtgtttgaatatatacttcattgtttaaggacaacatacaagtattttgctcttccacacaaaattacaaaatccagccttctaaagcctctgaatgcaattacatgtatttcagaacattctaaagaagtaataaatcatcatccagatgtacaaacaaaagatgataagctcaaaaactcagttttggcccaaggtcctggtgctaccagttcagctgcaaatacctgtaaggtacagccacttactcttaaagagactgctgaaagttttggaagcccaccaaaagaagaaatgggaaatgaacacatcagtgtccaccctgaaaactcagactgtatccaagcagatgttaactctgatgattacaagggtgataaagtataccatccagaaacaggaaggaaaaacgagaaagagaaagttggaaggaagggcaagcatctgttgactgttgatcagaaacgtggagagcatgttgtctgtggcagcacacgtaataatgagtcagagagcactttggatttagaaggcttccaaaatcccacagctaaagagtgtgagggacttgccactttagataacaaggctgatcttgatggagaaagtacagaaggtactgaggaactagaagactctctaaaccactttacccactcagtacagggccagacatcagaaatgattccctctgatgaagaggaggaggacgacgaagaagaggaggaggaagaagaacctaggctcaccattaaccaaagggaagatgaagatggcatggctaatgaagatgagttagacaacacctacactgggtcaggggatgaggacgccctatctgaagaggatgatgagttaggcgaagctgctaagtatgaagacgtgaaagaatgtggaaaacatgtagaaagagctctcctagtggaacttaataaaataagtctcaaggaagaaaatgtatgtgaagaaaaaaattcacctgtggatcagtctgattttttttatgaattcagtaaacttatcttcaccaaaggcaagtctcctacggtagtgtgcagcttatgcaaacgagagggtcatctaaagaaggactgtcctgaagacttcaaaagaatccagctagaacctctgccaccattaacacccaagtttttaaatatcttagatcaagtctgtatccagtgttataaggatttttctccaacaattatagaagatcaggctcgtgaacatattcggcaaaacctagaaagtttcataagacaggactttccaggaactaaattgagcctgtttggctcctccaaaaatggatttgggttcaaacagagtgaccttgacgtctgtatgacaattaatggacttgaaactgctgagggattggactgtgtcagaactattgaagaattagcaagagtcctcagaaaacattcaggtctgagaaacatcttacctattacaacagcaaaggtgccaattgtgaagttcttccatttgagaagtggtctggaagtagatatcagtttgtataacacattggcccttcataacacaaggcttttatctgcttattccgccattgatcccagagtgaagtatttgtgctataccatgaaagtatttacaaagatgtgtgatattggtgatgcatctagaggcagcttatcatcgtatgcatatactcttatggtgctatattttctccagcagaggaatccaccagtcattcctgtccttcaagagatatacaaaggtgaaaagaaacctgaaatatttgttgatggctggaatatttatttttttgatcaaatagatgaactgcctacctattggtcagaatgtggaaaaaatacagaatctgttgggcagttatggttgggccttcttcgtttctacacagaggaatttgattttaaagaacatgttattagcatcaggagaaaaagtctgcttacaacttttaagaaacagtggacctcaaaatacattgttattgaagatccctttgatttgaatcataatcttggagctggattatcaaggaaaatgacaaattttataatgaaggcttttatcaatggtagaagagtatttggtattcctgtcaagggatttccaaaggactacccctcaaaaatggaatacttttttgatccagatgtgttaactgaaggagagctggccccaaatgatagatgttgtcgaatttgtggaaaaatcggacacttcatgaaggactgtcctatgaggagaaaagtaagacggcggcgagatcaggaagatgccctgaaccaaagataccctgagaacaaggaaaaaagaagcaaagaggacaaagaaattcacaacaagtacacagaaagggaggtgtcaacaaaagaagataagcccatacagtgcacacctcagaaagccaagccaatgcgggcagctgctgacctggggagggagaagatcctcaggccaccagtagaaaaatggaagagacaggatgacaaagacttaagagaaaaacgttgttttatttgtggaagagaagggcacattaaaaaggaatgcccacagtttaaaggctcttcaggtagcctttccagtaaatatatgactcagggaaaagcctcagcgaagaggacccagcaggaatcatgagggaaggaaaatgcagcactctaaatggccactcaggcgttcctattcactcggaaaattaggttcatttcacaggacacagcagtgtagatcaggcttcaacttaacatttaagggaaatgtcagatttttttttaatttaatgaaattgttaatgaggaaaaatttttaatatagtcttatctaccacacatccccatagatttaaggattttaatagaaagacatgatgtatgtatttaagccacgttaaaagaaaaaatataactatggaccggtattcagtgaatacagtttcatggtttttaattctttcaaagcacattaaaaatggtgtgctgataaaccccaagtaaattaaccctttttccgtataaatccattttttgttttgaagaggggaaattatatttattgttgtttactgaatcctggtgtgaaagcatatcagatatgtatgaactgctactgctgtacttccgatttacggacatcattttattgctatttgtagacgtgataacatgaacatgagtacctatttatgtgggccttcagtggatgggcagtgccactcaggtctctggggtttccctctctaattttaagtaaattgacatataactactatgcttataaaaatgaagtaaggaaaacaagtagtcctgtttgccactaaaaacattttcaaaggaaaaataaaatgaaagtactttttactttttatgatactcagaaattaggatgaagaacttttaaaattgctgaagatcaaagaggttatctctgccagtcacaagtgtggctggtgtcattctgggtctgactggagccctcctggactgtttctttaatttcaaaagccctgcagacatagtacctggtcagaactatgcctcggtttatttatcattttgaaataaaatcagaatttcaacctgtaaaaaaaaaaaaaaaaaa

SEQ ID NO: 2 - protein, 1495 aa, Homo sapiens
MGDTAKPYFVKRTKDRGTMDDDDFRRGHPQQDYLIIDDHAKGHGSKMEKGLQKKKITPGNYGNTPRKGPCAVSSNPYAFKNPIYSQPAWMNDSHKDQSKRWLSDEHTGNSDNWREFKPGPRIPVINRQRKDSFQENEDGYRWQDTRGCRTVRRLFHKDLTSLETTSEMEAGSPENKKQRSRPRKPRKTRNEENEQDGDLEGPVIDESVLSTKELLGLQQAEERLKRDCIDRLKRRPRNYPTAKYTCRLCDVLIESIAFAHKHIKEKRHKKNIKEKQEEELLTTLPPPTPSQINAVGIAIDKVVQEFGLHNENLEQRLEIKRIMENVFQHKLPDCSLRLYGSSCSRLGFKNSDVNIDIQFPAIMSQPDVLLLVQECLKNSDSFIDVDADFHARVPVVVCREKQSGLLCKVSAGNENACLTTKHLTALGKLEPKLVPLVIAFRYWAKLCSIDRPEEGGLPPYVFALMAIFFLQQRKEPLLPVYLGSWIEGFSLSKLGNFNLQDIEKDVVIWEHTDSAAGDTGITKEEAPRETPIKRGQVSLILDVKHQPSVPVGQLWVELLRFYALEFNLADLVISIRVKELVSRELKDWPKKRIAIEDPYSVKRNVARTLNSQPVFEYILHCLRTTYKYFALPHKITKSSLLKPLNAITCISEHSKEVINHHPDVQTKDDKLKNSVLAQGPGATSSAANTCKVQPLTLKETAESFGSPPKEEMGNEHISVHPENSDCIQADVNSDDYKGDKVYHPETGRKNEKEKVGRKGKHLLTVDQKRGEHVVCGSTRNNESESTLDLEGFQNPTAKECEGLATLDNKADLDGESTEGTEELEDSLNHFTHSVQGQTSEMIPSDEEEEDDEEEEEEEEPRLTINQREDEDGMANEDELDNTYTGSGDEDALSEEDDELGEAAKYEDVKECGKHVERALLVELNKISLKEENVCEEKNSPVDQSDFFYEFSKLIFTKGKSPTVVCSLCKREGHLKKDCPEDFKRIQLEPLPPLTPKFLNILDQVCIQCYKDFSPTIIEDQAREHIRQNLESFIRQDFPGTKLSLFGSSKNGFGFKQSDLDVCMTINGLETAEGLDCVRTIEELARVLRKHSGLRNILPITTAKVPIVKFFHLRSGLEVDISLYNTLALHNTRLLSAYSAIDPRVKYLCYTMKVFTKMCDIGDASRGSLSSYAYTLMVLYFLQQRNPPVIPVLQEIYKGEKKPEIFVDGWNIYFFDQIDELPTYWSECGKNTESVGQLWLGLLRFYTEEFDFKEHVISIRRKSLLTTFKKQWTSKYIVIEDPFDLNHNLGAGLSRKMTNFIMKAFINGRRVFGIPVKGFPKDYPSKMEYFFDPDVLTEGELAPNDRCCRICGKIGHFMKDCPMRRKVRRRRDQEDALNQRYPENKEKRSKEDKEIHNKYTEREVSTKEDKPIQCTPQKAKPMRAAADLGREKILRPPVEKWKRQDDKDLREKRCFICGREGHIKKECPQFKGSSGSLSSKYMTQGKASAKRTQQES

SEQ ID NO: 3 - DNA, 966 bp, Artificial Sequence
  5′ RACE sequence for exons 2-6, Zcchc6
  misc_feature (721)..(721): n is a, c, g, or t
  misc_feature (891)..(891): n is a, c, g, or t
  misc_feature (930)..(930): n is a, c, g, or t
  misc_feature (932)..(932): n is a, c, g, or t
  misc_feature (935)..(936): n is a, c, g, or t
  misc_feature (958)..(959): n is a, c, g, or t
  misc_feature (964)..(966): n is a, c, g, or t
tgtatgctatacgaagtatcgatgcgatctgcgttcttcttctttggttttcgggacctgggacgacagctggaaactgaacatcaatgttcacgtcagagtctctgaagcccagtcggctacaggaggatccataaagcctaagagaacagtctggtagcttgtgccggaacacactttccatgacacgcttaatttctagtctctgatccaagttttcactgtgtaaaccaaactcctgcaccactctgtcaatggcgctgccaactgcatgtatctgggagggcgctggcgggggcagtgtggtgagcaattcttcctcctgcttttccttaaggttcttcttgtgcctcttctccttgatgtgcttatgaccacccgcaatggagtcaatcaaagcatcgcacagtttgcaggtatactttgctgtcgggcagtttcgtggtcgccttttcagcctgtcgatgcagtctctcttgagccgttcctcagcctgctggaggcccaacagctccttggttgaaaggacagactcgtcgatgacaggaccgtccagatctccatcctgctcactgtcctccgttctagtcctccgtggcttacgaggtctggaccgctgtttcttgttttcaggacttcctgcttccatttctgacatggcttctaggctgctgaggtctttcccccccccccccctctcacagctctgaagatctctccnactcctgccaccctgtaggcatcgtcactttcctgaaaggatttccttcctttgagcggctttatgacaggaaatcctaggtccgggcttgaactctctcccagcgtgtcctgcattgaccagcgagattcatcagcagtagccattttttattttggatctttgtggtnatcagttcatcccaagcaaggctgactggtaattgggtngnaanngcataaggaattgcctggcgannccccnnn

(19) United States
(12) Patent Application Publication
(10) Pub. No.: US 2019/0201489 A1
(43) Pub. Date: Jul. 4, 2019

(54) COMPOSITIONS AND METHODS FOR PREVENTING BONE LOSS AND/OR STIMULATING BONE HEALING

(71) Applicant: NORTHEAST OHIO MEDICAL UNIVERSITY, Rootstown, OH (US)

(72) Inventors: Fayez SAFADI, Rootstown, OH (US); Tariq HAQQI, Rootstown, OH (US)

(73) Assignee: NORTHEAST OHIO MEDICAL UNIVERSITY, Rootstown, OH (US)

(21) Appl. No.: 16/314,136

(22) PCT Filed: Jun. 28, 2017
(86) PCT No.: PCT/US2017/039679

§ 371 Date: Dec. 28, 2018

Related U.S. Application Data
(63) Continuation-in-part of application No. PCT/US2017/031535, filed on May 8, 2017.
(60) Provisional application No. 62/355,469, filed on Jun. 28, 2016.

(51) Int. Cl. A61K 38/17; C12N 15/113; A61K 47/42; A61P 19/00; A61K 35/28; A61K 31/713

(52) U.S. Cl. CPC A61K 38/1774; C12N 15/113; A61K 31/713; A61P 19/00; A61K 35/28; A61K 47/42

(57) ABSTRACT

In an aspect of the present application, compositions and methods are provided for preventing bone loss and/or stimulating bone healing in a subject in need thereof. Compositions can comprise a pharmaceutically acceptable carrier and a therapeutically effective amount of a zinc-finger CCHC domain-containing protein 6 (ZCCHC6) inhibitor. Methods can include administering a therapeutically effective amount of the ZCCHC6 inhibitor to the subject.

### RELATED APPLICATIONS

This application claims the benefit of U.S. Provisional Patent Application Ser. No. 62/355,469, filed Jun. 28, 2016, as well as PCT Application No. PCT/US2017/031535, filed May 8, 2017, the entirety of each of which is hereby incorporated by reference for all purposes.

### TECHNICAL FIELD

The present application relates generally to compositions and methods for bone healing and prevention of bone loss in aging and disease and, more particularly, compositions and methods that include one or more zinc-finger CCHC domain-containing protein 6 (ZCCHC6) inhibitors for preventing bone loss and/or stimulating bone healing.

### SEQUENCE LISTING

The instant application contains a Sequence Listing which has been submitted in ASCII format via EFS-Web and is hereby incorporated by reference in its entirety. Said ASCII copy, created Jun. 20, 2017, is named Compositions and Methods for Bone Healing_ST25, and is 22,791 bytes in size.

### BACKGROUND

Articular cartilage is a connective tissue with chondrocytes embedded in a matrix composed of a framework of collagens, proteoglycans, glycoproteins, etc., which act to stabilize the extracellular matrix. Distributed chondrocyte-matrix interactions play an important role during the initiation and progression of osteoarthritis (OA) or degenerative arthritis leading to the loss of cartilage and the joint function.

OA is a global disease that affects the whole joint and the sufferer's quality of life. OA has a complex etiology with many factors that contribute to an increased risk of OA, such as obesity, genetics, aging, and trauma to the joint. When clinically evident, OA is characterized by joint pain, tenderness, limitation of movement, crepitus, occasional effusion, and variable degrees of inflammation without systemic effects. OA pathology is now being recognized as driven by a pro-inflammatory component as high levels of inflammatory cytokines are present in the synovial fluid of patients with OA.

Clinical options in OA are currently limited to the use of non-steroidal anti-inflammatory drugs (NSAIDS) but ultimately the affected joint is replaced by the total joint arthroplasty (TJA) procedure. However, TJA is expensive and only carried out as a last resort. From the onset of disease, OA patients suffer acute pain and become disabled due to disease progression resulting in the loss of joint function. Furthermore, the disease is a multifactorial process that is impacted by aging, genetic predisposition, abnormal biomechanics, obesity, and trauma. Accordingly, there remains a significant need to develop an understanding OA so that additional approaches for treating OA and related diseases can be developed.

### SUMMARY

The present application relates generally to compositions and methods for bone healing and prevention of bone loss in aging and disease and, more particularly, compositions and methods that include one or more zinc-finger CCHC domain-containing protein 6 (ZCCHC6) inhibitors for preventing bone loss and/or stimulating bone healing.

ZCCHC6 (also known as TUT7), is a non-canonical poly(A) polymerase, has been shown to be important for the regulation of multiple functions in immune cells but its expression and function in bone is not known. The inventors of the present application investigated the expression and role of TUT7 in bone homeostasis in mice. Using TUT7 knockout mice, it was discovered that TUT7 was expressed in osteoblasts, osteoclasts but the expression decreased with age in bone. Mice with global TUT7 deficiency exhibit higher bone mineral density, cancellous bone volume, and enhanced osteoblast differentiation and function ex vivo compared to wild type littermates. Mineral apposition and bone formation rate were significantly high in TUT7 deficient mice. Osteoclast number and bone resorption were significantly reduced in TUT7KO mice. TUT7 knockout mice had low serum RANKL but increased expression of Osterix in osteoblasts. Importantly, reintroduction of TUT7 inhibited the Osterix expression and activity in TUT7KO osteoblasts. Based at least on these data, the inventor concluded that TUT7 is a novel, negative regulator of bone mass and acts through the inhibition of Osterix expression and activity in osteoblasts.

Based at least in part on the foregoing, the inventors of the present application investigated the potential of TUT7 as a potential novel target for therapeutic development (e.g., in bone formation). Data generated by the inventors clearly shows that inhibiting TUT7 leads to increased bone mass. Based on this discovery, the present application includes compositions and methods for inhibiting TUT7 to promote any one or combination of therapeutic effects including, but not limited to, accelerating fracture healing, inducing bone regeneration in large bony defects, prevention and/or treatment of osteoporosis and other osteopathy-related conditions (e.g., inflammation-induced bone loss associated with aging and rheumatoid arthritis), prevention and/or treatment of osteogenesis imperfect and osteomalacia, spinal fusion, and craniofacial re-construction of the mandible, maxilla, and cranial bones.

In an aspect of the present application, a composition for preventing bone loss and/or stimulating bone healing in a subject in need thereof can comprise a pharmaceutically acceptable carrier and a therapeutically effective amount of a ZCCHC6 inhibitor.

In another aspect of the present disclosure, a method of preventing bone loss and/or stimulating bone healing in a subject in need thereof can comprise administering a therapeutically effective amount of a ZCCHC6 inhibitor to the subject.

### BRIEF DESCRIPTION OF THE DRAWINGS

The foregoing and other features of the present application will become apparent to those skilled in the art to which the present application relates upon reading the following description with reference to the accompanying drawings, in which:

FIGS. 1A-H provide graphs and images showing that the absence of TUT7 expression has no severe effect on early postnatal development and skeletal morphology. (A) Differential mRNA expression of TUT7 in different tissues in WT mice as determined by SYBR-Green based qPCR. TUT7 was widely expressed in different tissues including bone. (B) Expression of TUT7 mRNA in aging long bone tissue in WT mice. TUT7 mRNA expression was highest during early postnatal skeletal development. (C) Generation of TUT7 KO mice was used by gene trapping. PCR of genomic DNA used to confirm wildtype (+/+; WT), heterozygous (+/−), and homozygous (−/−; TUT7KO). (D) Confirmation of the absence of TUT7 by qPCR in different tissues. (E-F) Absence of TUT7 had no effect on body weight or body length. Measurements of body weights (E) and lengths (F) of male and female WT and TUT7KO mice during different stages of development. (G-H) Genomic deletion of TUT7 had no apparent effect on skeletal morphology. Skeletal prep (G) of 5-day old WT and TUT7KO mice and X-ray analysis (H) of 8-week old WT and TUT7KO female mice. Data are expressed as means +/− SEM. ***P<0.001.

FIGS. 2A-H provide graphs and images showing that TUT7 deficiency enhances bone formation in vivo. (A) Absence of TUT7 had no effect on the lengths of WT and TUT7KO femurs at 4-, 8-, and 16-weeks of age. (B-C) Ablation of TUT7 results in enhanced bone mineral density (BMD; g/cm²) as shown in WT and TUT7KO male and female full body (B) and femur (C) by DEXA analysis. (D-H) Absence of TUT7 enhances trabecular bone mass compared to WT. MicroCT 3D-images (D) and analysis (E-H) of WT and TUT7KO male and female 8-week femurs. (E-H) Notice the increase in bone volume/tissue volume (BV/TV) (E), trabecular number (Tb.N) (F) and trabecular thickness (Tb.Th) (H) and decrease in trabecular separation (Tb.Sp.) (G) seen in the TUT7KO mice and compared to WT mice. Data are expressed as means +/−SEM. *P<0.05, **P<0.01.

FIGS. 3A-H provide graphs and images showing the absence of TUT7 expression increases bone matrix mineralization in vivo. (A-E) Histological images (A) and analyses (B) of femurs from 8-weeks old WT and TUT7KO mice. Deficiency of TUT7 expression results in enhanced trabecular number (Tb.N) and decreased trabecular separation (Tb.Sp.), but no difference in the number of osteoblasts (N.Ob/B.Pm) or osteoblast surface (Ob.S/BS) (B). (C-D) Dynamic histomorphometric calcein images (C) and analyses (D) of femurs from 8-weeks old WT and TUT7KO mice. Notice the significant increase in the mineral apposition rate (MAR) in TUT7KO mice and bone formation rate (BFR) compared to WT mice (D). (E) Osteocalcin levels in the serum of WT and TUT7KO mice determined by ELISA. (F) Expression of TUT7 in WT bone-marrow progenitor cells during osteoblast differentiation. TUT7 is most highly expressed during early osteoblastogenesis. (G-H) Absence of TUT7 enhances osteoblast differentiation. Bone-marrow progenitor cells from WT and TUT7KO mice were differentiated with osteogenic media or left undifferentiated, and terminated for ALP staining, activity, and qPCR (G) analyses. Furthermore, bone-marrow progenitor cells differentiated with osteogenic media were terminated for von Kossa staining and analyses (H). Scale bar; 200 um. Data are expressed as means +/−SEM. *P<0.05, ***P<0.01.

FIGS. 4A-G provide graphs and images showing that TUT7 deficiency inhibits osteoclast differentiation and resorption in vivo, but has no effect on osteoclastogenesis in vitro. (A-B) TRAP stained images (A) and histomorphometric analyses (B) of femurs from 16-weeks old WT and TUT7KO mice. Absence of TUT7 expression significantly decreased the number of osteoclasts per bone perimeter (N.Oc/B.Pm), but has no effect on the percentage of osteoclast surface per bone surface (Oc.S/BS) (B). (C-D) Biochemical analyses of osteoclast markers in serum of 16-weeks old WT and TUT7KO mice. Notice the significant reduction in CTX-1 (C), RANKL, and RANKL/OPG ratio, but no difference in the levels of OPG (D). TUT7 expression during osteoclastogenesis (E). TUT7 was not required for osteoclast differentiation in vitro. Osteoclast bone marrow progenitor cells from WT and TUT7KO mice were plated, differentiated, and terminated for TRAP staining and analysis (F). Ablation of TUT7 has no effect on osteoclast resorption activity in vitro. Osteoclast bone marrow progenitor cells (OCPs) from WT and TUT7KO mice were plated on Corning calcium phosphate discs, differentiated, and terminated to analyze resorption activity and to quantify the resorption area (G). Scale bar; 200 μm. Data are expressed as means +/−SEM. *P<0.05.

FIGS. 5A-F provide graphs and images showing that osteoblasts from TUT7KO mice inhibit osteoclastogenesis by inhibiting RANKL expression. (A-C) Co-culture assay and analyses of WT or TUT7KO mice-derived primary calvaria osteoblasts cultured with bone marrow-derived osteoclast progenitor cells from WT or TUT7KO mice. Note that in cultures containing TUT7KO-derived osteoblasts the number of WT mouse-derived osteoclasts was significantly reduced compared to co-cultures with WT osteoblasts as shown by TRAP staining (A), activity (B), and count (C). (D-E) TUT7 deficiency inhibits RANKL expression in osteoblasts, but not of OPG mRNA expression. Total RNA isolated from undifferentiated and differentiated WT and TUT7KO osteoblasts was used to examine the expression of RANKL (D) and OPG (E) mRNAs by qPCR. (F) Absence of TUT7 inhibits RANKL expression in long bone and calvaria of TUT7KO mice as determined by qPCR. Scale bar; 200 μm. Data are expressed as means +/−SEM. *P<0.05, **P<0.01, ***P<0.01.

FIGS. 6A-H provide graphs and images showing that TUT7 negatively regulates Osterix expression in osteoblasts. (A-B) Osteogenesis gene expression analyses in osteoblasts from WT and TUT7KO mice revealed a 7-fold increase in Osterix mRNA expression in the osteoblasts from TUT7KO mice and was verified by qPCR (C). Removal of TUT7 significantly enhances Osterix protein expression in osteoblasts (D). (E-F) Overexpression of TUT7 negatively regulates the expression of osteoblast markers in vitro. MC3T3-E1 osteoblast-like cells overexpressing TUT7 showed a significant reduction in Osterix mRNA expression and ALP and a significant increase in RANKL expression as determined by qPCR (E). Protein isolated from MC3T3-E1 osteoblast-like cells overexpressing TUT7 showed a significant reduction in the levels of Osterix protein by Western immunoblotting (F). (G-H) Overexpression of TUT7 negatively regulates Osterix activity in vitro. MC3T3-E1 osteoblast-like cells overexpressing TUT7 showed a significant reduction in Osterix activity determined using a Luciferase-based reporter vector (G). Primary osteoblasts from WT mice overexpressing TUT7 also resulted in a significant reduction in Osterix activity; however, when TUT7 expression was restored in TUT7KO osteoblasts, Osterix activity was downregulated and was similar to the levels in WT osteoblasts as determined by luciferase activity assay (H). Data are expressed as means +/−SEM. *P<0.05, **P<0.01.

FIG. 7 provides images showing chondrocytes present in the damaged human cartilage express IL-6 (arrows). IL-6 expressing chondrocytes were immunohistochemically localized using a mouse monoclonal antibody (sc-130326) and the VectaStain kit. IL-6 expressing chondrocytes were abundant in the damaged area (no Safranin-O staining). Control sections were incubated with mouse isotype control IgG only. SC-smooth cartilage, DC-damaged cartilage.

FIGS. 8A-C provide graphs and images showing IL-1β is a potent inducer of IL-6 and ZCCHC-6 expression in OA chondrocytes. Chondrocytes were stimulated with IL-1 β (5 ng/ml) for 24 h (A) IL-6 gene expression was determined using Taqman assay and is shown relative to the expression levels of housekeeping gene β-actin. (B) Secreted IL-6 was quantified by sandwich ELISA. (C) ZCCHC6 protein expression was determined using Western immunoblotting. Representative results are shown. Data are represented as Mean±SD(n=3). Each assay was run in duplicate. *p<0.005.

FIGS. 9A-B provide images and a graph showing ZCCHC6 knockdown inhibited IL-6 expression in IL-1 β-stimulated human chondrocytes. (A) SMARTpool siRNA-induced knockdown of ZCCHC6 was confirmed by immunoblotting. (B) Human chondrocytes transfected with non-targeting and ZCCHC6-targeting SMARTpool siRNAs were stimulated 48 h later with IL-1 β (1 ng/ml) for 3 h, 6 h, and 24 h and secreted IL-6 protein concentration in culture supernatants was determined by sandwich ELISA, n=3, *p<0.005, **p<0.001, ZCCHC6 siRNA transfected chondrocytes vs. chondrocytes transfected with native control siRNAs. NC=Negative Control.

### DETAILED DESCRIPTION

### Definitions

All scientific and technical terms used in the present application have meanings commonly used in the art unless otherwise specified. The definitions provided herein are to facilitate understanding of certain terms used frequently herein and are not meant to limit the scope of the present application.

In the context of the present application, the term “A” or “an” means herein one or more than one; at least one. Where the plural form is used herein, it generally includes the singular.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning.

“Comprised of” is a synonym of “comprising” (see above).

Use of the term “includes” is not intended to be limiting.

As used herein, the term “bone loss” can refer to any situation in which skeletal mass, substance or matrix or any component of the skeleton, such as calcium and phosphate, is decreased or a bone or tooth is lost, damaged, or weakened such as in terms of its ability to resist being broken. The term “bone loss” can also encompass any situation characterized by bone deterioration, bone degradation, bone degeneration, loss of bone mass, loss of bone density, and any combinations of these conditions. The term can also be used interchangeably with “bone resorption”. Non-limiting methods or assays for determining bone loss are known and can include μCT, DEXA and von Kossa staining (e.g., determining the level of osteocalcin, CTX-1, RANKL and/or OPG).

As used herein, the terms “bone healing”, “bone regeneration”, and “remodeling” can be used interchangeably and refer to a cellular process that occurs at the cellular level. When the process becomes unbalanced, bone mass decreases and bones may become brittle. Reference to promoting bone healing or enhancing bone regeneration by the present application can imply a rebalancing of bone remodeling in such a situation. Enhancing bone repair or regeneration can refer to increasing bone repair or regeneration beyond what would normally occur in the absence of treatment using the present compositions and methods. Enhancing bone repair can include increasing the rate of bone repair and the amount of bone repair that occurs over a given time. For example, enhancing bone repair can include increasing the rate or amount of bone repair by 10%, 20%, 30%, 40%, 50%, 60%, 70%, 80%, 90%, 100%, 150%, 200%, or more compared with the amount or rate of bone repair or regeneration that would occur in an untreated subject.

As used herein, the terms “prevent” or “preventing” when used in the context of bone loss or bone resorption can refer to the inhibition of removal of existing bone either from the mineral phase and/or the organic matrix phase through one or variety of cellular and molecular mechanisms including, for example, direct or indirect alteration of osteoclast formation or activity. Inhibition can include complete inhibition (e.g., 100%) or substantially complete inhibition (e.g., greater than about 50%, about 60%, about 70%, about 80%, about 90%, about 95%, or about 99%).

As used herein, the terms “stimulate” or “stimulating” when used in the context of bone healing can refer to promoting bone formation (e.g., by osteoblast proliferation). The term “promoting” in respect to bone regeneration can refer to the process of increasing the amount of bone tissue, bone cells, bone cell differentiation, bone matrix, etc., in a manner that allows bone regeneration. Thus, in some instances, promoting can refer to at least about 10%, 20%, 50%, 80%, 100%, or more increase in bone regeneration or at least about 10%, 20%, 50%, or 80% arrest or more in bone resorption. Those of skill in the art will understand that various methodologies and assays can be used to assess the promotion of bone regeneration or healing.

As used herein, the terms “zinc-finger CCHC domain-containing protein 6” or “ZCCHC6”, also known as Terminal Uridylyltransferase 7 (TUT7), PAPD6 (PAP associated domain containing 6) and HS2, can refer to a non-canonical poly(A) polymerase. ZCCHC6 is a 1,495 amino acid uridylyltransferase that mediates RNA uridylation. The gene that encodes ZCCHC6 maps to human chromosome 9q21.33 and mouse chromosome 13 B2. SEQ ID NO:1 provides the nucleotide sequence for Homo sapiens ZCCHC6 transcript variant 1 (mRNA). SEQ ID NO:2 provides the amino acid sequence for Homo sapiens ZCCHC6 protein.

As used herein, the term “ZCCHC6 inhibitor” can refer to any natural or synthetic compound, agent, moiety, or substance that inhibits and/or reduces the activity, function, secretion, expression, and/or a combination thereof, of ZCCHC6. Such compounds, agents, moieties, or substances can include, but are not limited to, small organic molecules, antisense nucleic acids, siRNA DNA aptamers, peptides, antibodies, non-antibody proteins, cytokines, chemokines, and chemo-attractants. In some embodiments, the inhibition is at least 20% (e.g., at least 50%, 70%, 80%, 90%, 95%, 98%, 99%, 99.5%, 100%) of the activity or function as compared to ZCCHC6 activity or function in the absence of the inhibitor. Assays that can be used (or adapted for use) to analyze ZCCHC6 activity and function are known in the art (see, e.g., Lin, S. and Gregory R I, RNA Biol., 2015; 12(8):792-800). Assays for analyzing ZCCHC6 secretion and expression are also known in the art and can include, for example, PCR, quantitative PCR and Western blotting. Non-limiting examples of ZCCHC6 inhibitors are discussed below.

As used herein, the term “activity” with reference to ZCCHC6 activity can refer to a cellular, biological, and/or therapeutic activity or function of ZCCHC6. One example of such activity can include, but is not limited to, negative regulation of bone mass via the inhibition of Osterix expression and activity in osteoblasts.

As used herein, the term “polynucleotide” can refer to oligonucleotides, nucleotides, or to a fragment of any of these, to DNA or RNA (e.g., mRNA, rRNA, tRNA) of genomic or synthetic origin which may be single-stranded or double-stranded and may represent a sense or antisense strand, to peptide nucleic acids, or to any DNA-like or RNA-like material, natural or synthetic in origin, including, e.g., iRNA, ribonucleoproteins (e.g., iRNPs). The term can also encompass nucleic acids, i.e., oligonucleotides, containing known analogues of natural nucleotides. Additionally, the term can encompass nucleic acid-like structures with synthetic backbones. The term can also encompass artificial or synthetic polynucleotides. Artificial or synthetic polynucleotides can include any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Other such modifications are well known to those of skill in the art.

As used herein, the term “polypeptide” can refer to an oligopeptide, peptide, polypeptide, or protein sequence, or to a fragment, portion, or subunit of any of these, and to naturally occurring or synthetic molecules. The terms “polypeptide” can also include amino acids joined to each other by peptide bonds or modified peptide bonds, i.e., peptide isosteres, and may contain any type of modified amino acids. Additionally, the term “polypeptide” can include peptides and polypeptide fragments, motifs and the like, glycosylated polypeptides, and all “mimetic” and “peptidomimetic” polypeptide forms.

As used herein, the term “subject” can refer to any warm-blooded organism including, but not limited to, human beings, rats, mice, dogs, goats, sheep, horses, monkeys, apes, pigs, rabbits, cattle, etc. When the term is used in the context of a subject needing or requiring compositions of the present application, the term may be referred to as “a subject in need thereof” and include subjects that have been clinically diagnosed (e.g., by a medical professional, e.g., a physician) as being in need of compositions of the present application, subjects that are suspected of being in need of compositions of the present application, subjects at risk for a disease or condition and who may benefit from compositions of the present application, and subjects that are already suffering from a disease or condition and who may benefit from compositions of the present application.

As used herein, the term “antibody” is used in the broadest sense and can include polyclonal antibodies, monoclonal antibodies, and epitope binding antibody fragments thereof so long as they exhibit the desired binding specificity.

As used herein, the term “epitope” can refer to that portion of any molecule capable of being recognized by, and bound by, an antibody. In general, epitopes consist of chemically active surface groupings of molecules, for example, amino acids or sugar side chains, and have specific three-dimensional structural characteristics as well as specific charge characteristics. The epitopes of interest for the present application are epitopes comprising amino acids.

As used herein, the terms “monoclonal antibody” or “monoclonal antibodies” can refer to a preparation produced by one type of immune cell that are all clones of a single parent cell typically including identical antibodies directed against a single epitope. The modifier “monoclonal” indicates the character of the antibody and is not to be construed as requiring production of the antibody by any particular method.

As used herein, the terms “polyclonal antibody” or “polyclonal antibodies” can refer to a preparation typically including different antibodies directed against multiple epitopes. The modifier “polyclonal” indicates that character of the antibody as being obtained from a heterogeneous population of antibodies, and is not to be construed as requiring production of the antibody by any particular method.

As used herein, the term “small molecule” can refer to any organic molecule or unimolecular biologic. The term can refer to a molecule of a size comparable to those organic molecules generally used in pharmaceuticals. The term excludes biological macromolecules (e.g., proteins, nucleic acids, etc.). Small organic molecules can range in size up to about 5000 Da, up to 2000 Da, or up to about 1000 Da.

As used herein, the term “effective route” can refer to a route that provides for delivery of a composition of the present application to a desired compartment, system, or location. For example, an effective route is one through which a composition of the present application can be administered to provide at a desired site of action (e.g., a site of bone damage) an amount of the composition sufficient to effectuate a beneficial or desired clinical result (e.g., preventing bone loss and/or stimulating bone healing).

As used herein, the term “therapeutically effective amount” can refer to the amount of a composition of the present application determined to produce a therapeutic response in a subject. For example, compositions of the present application may prevent bone loss and/or stimulate bone healing in a subject in need thereof. Such therapeutically effective amounts are readily ascertained by one of ordinary skill in the art. Thus, to “treat” means to deliver such an amount.

As used herein, the terms “treat,” “treating,” or “treatment” are used broadly in relation to the present application and each such term can encompass, among others, ameliorating, inhibiting, or curing a bone deficiency, bone dysfunction, bone disease, or other deleterious process, including those that interfere with and/or result from a therapy. Non-limiting examples of such deficiencies, dysfunction, and disease can include bone fractures and breaks, bone defects caused by trauma or congenital conditions, osteoporosis and other osteopathy-related conditions (e.g., inflammation-induced bone loss associated with aging and rheumatoid arthritis), osteogenesis imperfect and osteomalacia, spinal fusion, and craniofacial re-construction of the mandible, maxilla, and cranial bones.

As used herein, the term “pharmaceutical composition” can refer to a preparation of one or more of the active ingredients or agents described herein (e.g., a ZCCHC6 inhibitor) with other components, such as physiologically suitable carriers and excipients. The purpose of a pharmaceutical composition is to facilitate administration of one or a combination of active ingredients or agents to a subject in need thereof.

As used herein, the term “biocompatible” can refer to any material that does not cause injury or death to a subject or induce an adverse reaction in a subject when placed in contact with the subject's tissues. Adverse reactions include for example inflammation, infection, fibrotic tissue formation, cell death, or thrombosis. The terms “biocompatible” and “biocompatibility” when used herein are art-recognized and mean that the material is neither itself toxic to a subject, nor degrades (if it degrades) at a rate that produces byproducts (e.g., monomeric or oligomeric subunits or other byproducts) at toxic concentrations, does not cause prolonged inflammation or irritation, or does not induce more than a basal immune reaction in the host.

As used herein, the term “mesenchymal stem cell” or “MSC” can refer to cells that are derived from the embryonal mesoderm and can be isolated from many sources, including adult bone marrow, peripheral blood, fat, placenta, and umbilical blood, among others. MSCs can differentiate into many mesodermal tissues, including muscle, bone, cartilage, fat, and tendon. There is considerable literature on these cells. See, for example, U.S. Pat. Nos. 5,486,389; 5,827,735; 5,811,094; 5,736,396; 5,837,539; 5,837,670; and 5,827,740. See also Pittenger, M. et al, Science, 284:143-147 (1999).

### Compositions

One aspect of the present application can include a composition for preventing bone loss and/or stimulating bone healing in a subject in need thereof. The composition can comprise a pharmaceutically acceptable carrier and a therapeutically effective amount of a ZCCHC6 inhibitor.

Any means/agent including, but not limited to, chemical (e.g., a chemical compound, including but not limited to a pharmaceutical, drug, small molecule), protein (e.g., anti-ZCCHC6 antibody), peptide, microorganism, biologic, nucleic acid (including genes coding for recombinant proteins or antibodies as well as anti-sense molecules, such as siRNAs), or genetic constructs (e.g., vectors, such as expression vectors, including but not limited to expression vectors which lead to expression of an antagonist against ZCCHC6 activity) can be used to inhibit ZCCHC6 function and/or activity.

In one example, the ZCCHC6 inhibitor can comprise a small molecule. Candidate ZCCHC6 inhibitors may be tested in animal models. Typically, the animal model is one for the study of bone disease or degeneration. The study of various bone diseases in animal models (for instance, mice) is a commonly accepted practice for the study of such diseases (e.g., Destabilization of Medial Meniscus (DMM), Anterior Cruciate Ligament Transection (ACLT), and Ovirectomy). Results are typically compared between control animals treated with candidate agents and the control littermates that did not receive treatment. Transgenic animal models are also available and are commonly accepted as models for human disease. Candidate agents can be used in these animal models to determine if a candidate agent prevents bone loss and/or stimulates bone healing.

In one example, the ZCCHC6 inhibitor can include a human ZCCHC6 antibody or active fragment thereof. The human ZCCHC6 antibody, or active fragment thereof, can be reactive against at least one epitope of a ZCCHC6 protein having SEQ ID NO:2. Human ZCCHC6 antibodies, which may also be known as a PAPD6 or Hs2 antibodies, are commercially available from Proteintech Cat #25196-1-AP.

Also within the scope of the present application is the production and use of polyclonal or monoclonal antibodies, or active fragments thereof, which recognize one or more antigenic portion(s) of ZCCHC6 including, but not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above, which recognize one or more antigenic portion(s) of ZCCHC6. Antibodies directed against ZCCHC6 may be used to specifically inhibit ZCCHC6 function and/or activity.

All antibody molecules belong to a family of plasma proteins called immunoglobulins, whose basic building block, the immunoglobulin fold or domain, is used in various forms in many molecules of the immune system and other biological recognition systems. A typical immunoglobulin has four polypeptide chains, containing an antigen binding region known as a variable region and a non-varying region known as the constant region.

Native antibodies and immunoglobulins are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains.

Depending on the amino acid sequences of the constant domain of their heavy chains, immunoglobulins can be assigned to different classes. There are at least five major classes of immunoglobulins: IgA, IgD, IgE, IgG and IgM, and several of these may be further divided into subclasses (isotypes), e.g., IgG-1, IgG-2, IgG-3 and IgG-4; IgA-1 and IgA-2. The heavy chains constant domains that correspond to the different classes of immunoglobulins are called alpha (α), delta (β), epsilon (ε), gamma (γ) and mu (μ) respectively. The light chains of antibodies can be assigned to one of two clearly distinct types, called kappa (κ) and lambda (λ), based on the amino sequences of their constant domain. The subunit structures and three-dimensional configurations of different classes of immunoglobulins are well known.

The term “variable” in the context of variable domain of antibodies, refers to the fact that certain portions of the variable domains differ extensively in sequence among antibodies. The variable domains are for binding and determine the specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains.

The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity.

An antibody that is contemplated for use in the present application thus can be in any of a variety of forms, including a whole immunoglobulin, an antibody fragment, such as Fv, Fab, and similar fragments, a single chain antibody that includes the variable domain complementarity determining regions (CDR), and the like forms, all of which fall under the broad term “antibody,” as used herein. The present application contemplates the use of any specificity of an antibody, polyclonal or monoclonal, and is not limited to antibodies that recognize and immunoreact with a specific epitope.

The term “active fragment” can refer to a portion of a full-length antibody, generally the antigen binding or variable region. Examples of active fragments include Fab, Fab', F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments, which are capable of cross-linking antigen, and a residual other fragment (which is termed pFc′). Additional fragments can include diabodies, linear antibodies, single-chain antibody molecules, and multispecific antibodies formed from antibody fragments. As used herein, “functional fragment” with respect to antibodies, can refer to Fv, F(ab) and F(ab′)₂ fragments.

The present application further contemplates human and humanized forms of non-human (e.g., murine) antibodies. Such humanized antibodies can be chimeric immunoglobulins, immunoglobulin chains or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a nonhuman species (donor antibody), such as mouse, rat or rabbit having the desired specificity, affinity and capacity.

In another example, the ZCCHC6 inhibitor can include any polynucleotide by which the expression of a target gene (e.g., Zcchc6) is selectively inhibited. Using RNA interference (RNAi), for example, a mediator of sequence-specific mRNA degradation (e.g., a 19 to 23-nucleotide small interfering RNA) can be produced from a longer dsRNA by digestion with ribonuclease III. A cytoplasmic RISC (RNA-induced silencing complex) binds to an siRNA and directs degradation of an mRNA comprising a sequence complementary to one strand of the siRNA. The application of RNA interference in mammals has a therapeutic gene silencing effect.

In some instances, anti-sense oligonucleotides, including anti-sense RNA molecules, and anti-sense DNA molecules, can be used that act to directly block the translation of ZCCHC6 mRNA by binding thereto and thus preventing protein translation or increasing mRNA degradation, thus decreasing the level of ZCCHC6 proteins, and thus activity, in a cell. For example, antisense oligonucleotides of at least about 15 bases and complementary to unique regions of the mRNA transcript sequence encoding for ZCCHC6 (such as SEQ ID NO:1) may be synthesized, e.g., by conventional phosphodiester techniques. Methods for using antisense techniques for specifically inhibiting gene expression of genes whose sequence is known are well known in the art (e.g., see U.S. Pat. Nos. 6,566,135; 6,566,131; 6,365,354; 6,410,323; 6,107,091; 6,046,321; and 5,981,732).

In some instances, small inhibitory RNAs (siRNAs) can also function as inhibitors of expression of ZCCHC6 for use in the present application. Zcchc6 gene expression can be reduced by contacting a cell with a small double-stranded RNA (dsRNA), or a vector or construct causing the production of a small double stranded RNA, such that expression of ZCCHC6 is specifically inhibited (i.e., RNA interference or RNAi). Methods for selecting an appropriate dsRNA or dsRNA-encoding vector are well known in the art for genes whose sequence is known (e.g., see International Patent Publication Nos. WO 01/36646, WO 99/32619, and WO 01/68836).

In some instances, ribozymes can also function as inhibitors of ZCCHC6 expression for use in the present application. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Specific ribozyme cleavage sites within any potential RNA target are initially identified by scanning the target molecule for ribozyme cleavage sites, which typically include the following sequences, GUA, GuU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Antisense oligonucleotides, siRNA oligonucleotides, and ribozymes useful as inhibitors of expression of ZCCHC6 can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters.

Antisense oligonucleotides, siRNA oligonucleotides, and ribozymes of the present application may be delivered alone or in association with a vector. In its broadest sense, a “vector” is any vehicle capable of facilitating the transfer of the antisense oligonucleotide, siRNA oligonucleotide or ribozyme nucleic acid to cells. Preferably, the vector transports the nucleic acid to cells with reduced degradation relative to the extent of degradation that would result in the absence of the vector. In general, the vectors useful in the present application include, but are not limited to, plasmids, phagemids, viruses, other vehicles derived from viral or bacterial sources that have been manipulated by the insertion or incorporation of the antisense oligonucleotide, siRNA oligonucleotide or ribozyme nucleic acid sequences.

Methods for delivering siRNAs, ribozymes and/or antisense oligonucleotides into cells are well known in the art and include, but are not limited to, transfection, electroporation, microinjection, lipofection, calcium phosphate mediated transfection or infection with a viral vector containing the gene sequences, cell fusion, chromosome-mediated gene transfer, microcell-mediated gene transfer, spheroplast fusion, etc. Numerous techniques are known in the art for the introduction of foreign genes into cells and may be used in accordance with the present application, provided that the necessary developmental and physiological functions of the recipient cells are not disrupted. The technique may provide for the stable transfer of the gene to the cell, so that the gene is expressible by the cell, heritable and expressible by its cell progeny. Usually, the method of transfer includes the transfer of a selectable marker to the cells. The cells are then placed under selection to isolate those cells that have taken up and are expressing the transferred gene. Those cells are then delivered to a subject. A variation of the technique may provide for transient transfer of oligonucleotides or oligonucleotide coding genes to cells to enable temporary expansion of cells ex vivo or in vivo without permanent genetic modification.

In one example, the ZCCHC6 inhibitor can include a human siRNA directed against SEQ ID NO:1. Human ZCCHC6 siRNAs, also known as PAPD6 or Hs2 siRNAs, are commercially available from GE Healthcare Dharmacon Cat #LU-026009-0-0002, Sigma Chemical Company Cat #SASI_Hs02_00356991.

In another example, expression of ZCCHC6 may be inhibited by compounds (e.g., small molecules) acting on promoter activity, RNA processing or protein stability. In other instances, inhibition of the activity of ZCCHC6 may be achieved by using mutated ZCCHC6 polypeptides that compete with the wild-type ZCCHC6.

In another aspect, active agents of the present application (e.g., ZCCHC6 inhibitors) can be formulated as a pharmaceutical composition. Such pharmaceutical compositions can be formulated with other components, such as physiologically suitable carriers and excipients. A carrier (or diluent) can include any agent, compound, or moiety that does not cause significant irritation to a subject and does not abrogate the biological activity and properties of the administered active agents. Examples of acceptable carriers that are useful in the context of the present application include, without limitation, emulsions, creams, aqueous solutions, oils, ointments, pastes, gels, lotions, milks, foams, suspensions and powders. Acceptable carriers can further include, for example, a thickener, an emollient, an emulsifier, a humectant, a surfactant, a suspending agent, a film forming agent, a foam building agent, a preservative, an antifoaming agent, a fragrance, a lower monoalcoholic polyol, a high boiling point solvent, a propellant, a colorant, a pigment or mixtures thereof.

Excipients that may be used to formulate the compositions of the present application can include an inert substance that facilitates administration of the active ingredients. Examples, without limitation, of excipients include calcium carbonate, calcium phosphate, various sugars and types of starch, cellulose derivatives, gelatin, vegetable oils, and polyethylene glycols.

The compositions of the present application may be manufactured by processes well known in the art, e.g., by means of conventional mixing, dissolving, granulating, dragger-making, levigating, emulsifying, encapsulating, entrapping or lyophilizing processes.

In some instances, the pharmaceutically acceptable carrier can include a nanoparticle. Suitable nanoparticles can include any carrier structure which is biocompatible with and sufficiently resistant to chemical and/or physical destruction by the environment of use such that a sufficient amount of the nanoparticles remain substantially intact after injection into the blood stream, given intraperitoneally or orally or incubated with an in vitro sample so as to be able to reach the nucleus of a cell or some other cellular structure. If the drug can enter the cell in the form whereby it is adsorbed to the nanoparticles, the nanoparticles must also remain sufficiently intact to enter the cell. Biodegradation of the nanoparticle is permissible upon entry of a cell's nucleus. Nanoparticles can be solid colloidal particles ranging in size from 1 to 1000 nm. Nanoparticle can have any diameter less than or equal to 1000 nm, including 5, 10, 15, 20, 25, 30, 50, 100, 500 and 750 nm. ZCCHC6 inhibitors or other relevant materials can be incubated with the nanoparticles, and thereby be adsorbed or attached to the nanoparticle. Examples of nanoparticles suitable for drug delivery are known in the art and disclosed, for example, in U.S. Patent Publication Nos. 2003/0147966, 2014/0005258, and 2010/0034735.

In other instances, the pharmaceutically acceptable carrier can include a biocompatible material. Suitable biocompatible materials can include biocompatible polymers, graft materials, such as an allograft or xenograft, bone-derived materials, collagen (e.g., a collagen sponge), and biocompatible inorganic materials. Examples of biocompatible polymers can include polymers include natural or synthetic polymers such as polystyrene, polylactic acid, polyketal, butadiene styrene, styreneacrylic-vinyl terpolymer, polymethylmethacrylate, polyethylmethacrylate, polyalkylcyanoacrylate, styrene-maleic anhydride copolymer, polyvinyl acetate, polyvinylpyridine, polydivinylbenzene, polybutyleneterephthalate, acrylonitrile, vinylchloride-acrylates, polycaprolactone, poly(alkyl cyanoacrylates), poly(lactic-co-glycolic acid), and the like.

In one example, the biocompatible material is an inorganic material, such as an inorganic ceramic and/or bone substitute material. Exemplary inorganic materials or bone substitute materials include but are not limited to aragonite, dahlite, calcite, amorphous calcium carbonate, vaterite, weddellite, whewellite, struvite, urate, ferrihydrate, francolite, monohydrocalcite, magnetite, goethite, dentin, calcium carbonate, calcium sulfate, calcium phosphosilicate, sodium phosphate, calcium aluminate, calcium phosphate, hydroxyapatite, α-tricalcium phosphate, dicalcium phosphate, β-tricalcium phosphate, tetracalcium phosphate, amorphous calcium phosphate, octacalcium phosphate, BIOGLASS™, fluoroapatite, chlorapatite, magnesium-substituted tricalcium phosphate, carbonate hydroxyapatite, substituted forms of hydroxyapatite (e.g., hydroxyapatite derived from bone may be substituted with other ions such as fluoride, chloride, magnesium sodium, potassium, etc.), coral, silicate or silicate derived materials, or combinations or derivatives thereof.

In another example, the biocompatible material may comprise particles of bone-derived materials. The bone-derived material may include one or more of non-demineralized bone particles, demineralized bone particles, lightly demineralized bone particles, and/or deorganified bone particles.

In another example, the biocompatible material is a biodegradable polymer. Examples of biodegradable polymers include polylactide polymers include poly(D,L-Lactide)s; poly(lactide-co-glycolide) (PLGA) copolymers; polyglycolide (PGA) and polydioxanone; caprolactone polymers; chitosan; hydroxybutyric acids; polyanhydrides and polyesters; polyphosphazenes; and polyphosphoesters.

Functionalized poly(D,L-Lactide)s can also be used as biodegradable polymers. Examples of functionalized poly(D,L-Lactide)s include poly(L-lactide), acrylate terminated; poly(L-lactide), amine terminated; poly(L-lactide), azide terminated; poly(L-lactide), 2-bromoisobutyryl terminated; poly(L-lactide), 2-bromoisobutyryl terminated; poly(L-lactide) 4-cyano-4-[(dodecylsulfanylthiocarbonyl)sulfanyl]pentonate; poly(L-lactide) N-2-hydroxyethylmaleimide terminated; poly(L-lactide) 2-hydroxyethyl, methacrylate terminated; poly(L-lactide), propargyl terminated; poly(L-lactide), and thiol-terminated polymers.

Other biodegradable polymers that can be used include AB diblock copolymers, such as poly(ethylene glycol) methyl ether-block-poly(D,L lactide); poly(ethylene glycol) methyl ether-block-poly(lactide-co-glycolide) PEG; poly(ethylene glycol)-block-poly(ε-caprolactone) methyl ether PEG; and Polypyrrole-block-poly(caprolactone). Further biodegradable polymers include ABA triblock copolymers such as polylactide-block-poly(ethylene glycol)-block-polylactide PLA; poly(lactide-co-glycolide)-block-poly(ethylene glycol)-block-poly(lactide-co-glycolide); poly(lactide-co-caprolactone)-block-poly(ethylene glycol)-block-poly(lactide-co-caprolactone); polycaprolactone-block-polytetrahydrofuran-block-polycaprolactone; and polyglycolide-block-poly(ethylene glycol)-block-polyglycolide PEG.

Biodegradable polymers also include various natural polymers. Examples of natural polymers include polypeptides including those modified non-peptide components, such as saccharide chains and lipids; nucleotides; sugar-based biopolymers such as polysaccharides; cellulose; chitosan, carbohydrates and starches; dextrans; lignins; polyamino acids; adhesion proteins; lipids and phospholipids (e.g., phosphorylcholine).

In some instances, the biocompatible material is configured as a tissue scaffold. A tissue scaffold is a support structure that provides a matrix for cells to guide the process of bone tissue formation in vivo. The morphology of the scaffold guides cell migration and cells are able to migrate into or over the scaffold, respectively. The cells then are able to proliferate and synthesize new tissue and form bone and/or cartilage. While there are many criteria for an ideal tissue scaffold for bone tissue repair, an important characteristic is the presence of a highly interconnected porous network with both pore sizes and pore interconnections large enough for cell migration, fluid exchange, and eventual tissue in-growth and vascularization.

The biocompatible material can be molded or otherwise shaped during preparation to have any desired configuration as a tissue scaffold. Typically, the biocompatible material is molded to have the shape of the bone or bone-like material that it is being substituted for. However, the scaffold material can also be used for cosmetic work or “bioengineering,” where a support structure is provided for the creation of new tissue rather than the replacement or regeneration of existing tissue. In some instances, the tissue scaffold may be seeded with harvested bone cells and/or bone tissue, such as for example, cortical bone, autogenous bone, allogenic bones and/or xenogenic bone. In other instances, the tissue scaffold can be bioresorbable. For further information regarding suitable tissue scaffolds for bone repair or regeneration, see, for example, U.S. patent applications Ser. Nos. 11/793,625, 12/193,794, 13/908,627, and 14/216,451.

In another aspect, MSCs can be formulated for delivery with a ZCCHC6 inhibitor, formulated for co-administration with a composition comprising a ZCCHC6 inhibitor, or sequentially delivered before and/or after administration of a composition comprising a ZCCHC6 inhibitor.

Compositions of the present application can be formulated differently depending, for example, on the intended route of administration. Non-limiting examples of routes of administration can injection (e.g., intraosteal or intraoral), minimally invasive, and open surgical routes.

### Methods

Another aspect of the present application can include a method for preventing bone loss and/or stimulating bone healing in a subject in need thereof. The method can comprise administering a therapeutically effective amount of a ZCCHC6 inhibitor to the subject. In some instances, the ZCCHC6 inhibitor is formulated with a pharmaceutically acceptable carrier (such as those described above) as a pharmaceutical composition.

Depending on the subject, the composition can be administered via an effective route, such as injection, as discussed below.

The composition can be administered to the subject by another person, e.g., a healthcare provider according to a prescribed treatment protocol (e.g., as determined by a healthcare professional).

In some instances, the method of the present application can include in vivo placement of a composition, as described herein, for bioengineering, restoring or regenerating bone. In particular aspects of the method, bioengineering, restoring or regenerating bone is in vitro or ex vivo, including placement under body fluid conditions. The method includes positioning a composition to promote bone healing and/or prevent bone loss at or near the site in need of repair (e.g., for dental and orthopedic implants, craniomaxillofacial applications and spinal grafting).

Administering the composition can include contacting a site in need of bone repair or regeneration in a subject with the composition of the present application.

“Contacting”, as used herein, can refer to causing two items to become physically adjacent and in contact, or placing them in an environment where such contact will occur within a short timeframe. For example, contacting a site with a composition comprising a ZCCHC6 inhibitor includes administering the composition to a subject in need thereof at or near a site such that the ZCCHC6 inhibitor will interact with the site to prevent bone loss and/or stimulate bone healing. In some instances, the step of contacting the site comprises surgically implanting the composition. Methods of surgically implanting orthopedic implants and biocompatible materials for bone repair and regeneration are known to those skilled in the art.

In some instances, the composition is in an injectable form, and the step of contacting the site comprises administering the composition by injection to the site in need of bone repair or regeneration. “Injectable” refers to the ability of certain compositions of the present application to be introduced at an implant site under pressure (as by introduction using a syringe). An injectable composition of the present application may, for example, be introduced between elements or into a confined space in vivo (i.e., between pieces of bone or into the interface between a prosthetic device and bone, among others). For example, the compositions may be injected into the vertebral body for prevention or treatment of spinal fractures, injected into long bone or flat bone fractures to augment the fracture repair or to stabilize the fractured fragments, or injected into intact osteoporotic bones to improve bone strength.

Examples of injectable forms include a fluid injectable gel and a fluid injectable paste. A wide variety of flowable compositions suitable for injection are known to those skilled in the art, including various hydrogel compositions. See, for example, U.S. Pat. No. 8,309,106. Preferably, the injectable composition is extrudable through a syringe and/or a syringe having at an appropriate gauge needle coupled there to (e.g., at least a 13 gauge tube/needle).

A bone (i.e., bone tissue) is a rigid organ that constitutes part of the vertebral skeleton. Bone tissue includes two basic types—cortical (the hard, outer layer of bone) and cancellous bone (the interior trabecular or spongy bone tissue), which gives it rigidity and a coral-like three-dimensional internal structure. Other types of tissue found in bone include marrow, endosteum, periosteum, nerves, blood vessels and cartilage. Bone is an active tissue composed of different cells. Osteoblasts are involved in the creation and mineralization of bone; osteocytes and osteoclasts are involved in the reabsorption of bone tissue. The mineralized matrix of bone tissue has an organic component mainly of collagen and an inorganic component of bone mineral made up of various salts

Compositions of the present application can be used to repair or regenerate any type of bone. There are five types of bones in the human body. These are long bones, short bones, flat bones, irregular bones and sesmoid bones. Examples of long bones include the femur, the humerus and the tibia. Examples of short bones include carpals and tarsals in the wrist and foot. Examples of flat bones include the scapula, the sternum, the cranium, the os coxae, the pelvis, and ribs. Irregular bones are those which do not fit within the other categories, and include vertebrae, sacrum and mandible bones. Sesmoid bones are typically short or irregular bones, imbedded in a tendon, such as the patella. While not formally considered bone, teeth are also included in the definition of bone used herein.

The present application provides compositions and methods for preventing bone loss and/or stimulating bone healing. Bone injury requiring the methods of the present application can occur as a result of disease, chronic stress, or physical trauma. Examples of different types of bone injury include degenerative disc, cervical spondylosis, and bone fracture. Bone regeneration or healing is also called remodeling and occurs at the cellular level. When the process becomes unbalanced, bone mass decreases and bones may become brittle. Reference to promoting bone healing or enhancing bone regeneration by the present application implies a rebalancing of bone remodeling in such a situation. Enhancing bone repair or regeneration refers to increasing bone repair or regeneration beyond what would normally occur in the absence of treatment using the present compositions and methods. Enhancing bone repair includes increasing the rate of bone repair and the amount of bone repair that occurs over a given time. For example, enhancing bone repair includes increasing the rate or amount of bone repair by 10%, 20%, 30%, 40%, 50%, 60%, 70%, 80%, 90%, 100%, 150%, 200%, or more compared with the amount or rate of bone repair or regeneration that would occur in an untreated subject.

The method of the present application preferably occurs under aseptic conditions. “Aseptic”, as the term is used herein, can refer to methods to control or reduce the microbial bioburden in an environment. Tissues processed “aseptically” are tissues processed using sterile instruments, and special environmental surroundings (including for example “clean room technologies”).

The following examples are for the purpose of illustration only and are not intended to limit the scope of the claims, which are appended hereto.

### Example 1

In this study, we investigated the role of TUT7 in skeletal development and remodeling. We generated TUT7 knockout (KO) mice and characterized their skeletal phenotype. We discovered that deletion of TUT7 was not lethal and neonatal TUT7KO mice developed normally similar to wildtype (WT) littermates; however, postnatally TUT7KO mice have significantly increased bone mass compared to WT. Ex vivo analysis of bone marrow-derived osteoprogenitor cells demonstrated an increase in osteoblast differentiation and mineralization suggesting that TUT7 plays an important role in OB function. Interestingly, osteoclasts derived from TUT7KO mice ex vivo showed no significant difference in differentiation and function. Gene expression analysis using a PCR-based array showed that constitutive expression of master transcription factor Osterix (Osx) was significantly upregulated in OBs derived from TUT7KO mice compared to the expression levels in OBs from WT littermates. Furthermore, TUT7 overexpression in WT osteoblasts resulted in suppression of Osx expression and activity while absence of TUT7 resulted in enhanced Osx expression and activity. Overall, our data suggests that TUT7 is a negative regulator of bone formation through suppression of Osx in OBs. Taken together, this study is the first to demonstrate the role of TUT7 in postnatal skeletal development and remodeling.

### Materials and Methods

Cell Culture and Reagents. Minimum essential medium alpha (α-MEM) was purchased from Mediatech (Manassas, Va.). The preosteoblast-like cell line MC3T3-E1 subclone 4 was from ATCC (Manassas, Va.). Alcian Blue, Alizarin Red, Silver Nitrate, Sodium Thiosulfate, Fast Green, Sodium Tartrate, Sodium Acetate, Trypsin, Penicillin-Streptomycin, Amphotericin-B and Ascorbic Acid were from Thermo Fisher Scientific (Waltham, Mass.); β-glycerophosphate, Dexamethasone, Sodium Carbonate, Collagenase B and Fast Red Violet were from Sigma-Aldrich (St. Louis, Mo.). Toluidine blue was from Electron Microscopy Sciences (Hatfield, Pa.). The RANKL and M-CSF were from R&D systems (Minneapolis, Minn.). Osterix antibody was from Abcam (Cambridge, UK). GAPDH antibody was from Cell Signaling (Danvers, Mass.). TUT7 antibody and Calcein were from Santa Cruz Biotechnology (Dallas, Tex.). PCR Primers were purchased from IDT (Coralville, Iowa). The pcDNA3 expression vector was purchased from Invitrogen (Carlsbad, Calif.). pcDNA3-FLAG-mTUT7 was a gift from Zissimos Mourelatos (Addgene Plasmid #60044). pGL3-Basic luciferase vector was purchased from Promega (Madison, Wis.). The pGL3-osterix luciferase reporter construct was a kind gift from Dr. Mark Nanes (Emory University).

Generation of TUT7KO Mice. Mouse embryonic stem (ES) cell line (AEO325) containing a gene-trap insertion in the Zcchc6 gene (MMRRC/KOMP, University of California-Davis) was used to produce heterozygous Zcchc6 KO mice. The gene-trap genomic insertion site was located within introns 2-6. All known conserved protein motifs and domains in Zcchc6/TUT7 are downstream of the insertion site. Appropriate targeting by 5′ and 3′ homology arms was confirmed by PCR. Rapid amplification of C-DNA ends (RACE) demonstrated the presence of Exons 2-6 of Zcchc6 (SEQ ID NO: 3) in the ES cells. TUT7KO mice were generated by crossing heterozygous mice and displayed normal Mendelian ratio.

Skeletal Preparations. Whole skeletal preparations from day 5 newborn mice were prepared essentially as previously described. Wassersug R J, Stain Technol 51(2):131-134 (1976). Images were taken using a Nikon SMZ 800 stereoscope (Nikon, Melville, N.Y.).

Micro-CT Analysis. Femurs from 8- and 16-weeks old male and female WT and TUT7KO mice (n≥5) were analyzed using a SkyScan 1172 high-resolution microtomography (MicroCT) system (Bruker, Billerica, Mass.) as previously described (Abdelmagid et al., The American journal of pathology 184(3):697-713 (2014)). Scanned images were reconstructed using the NRecon software. Following reconstruction, samples were analyzed using the CTAn software. The trabecular regions of interest were taken 400 μm below the growth plate and extended 5,700-6,000 μm depending on age proximally towards the diaphysis. Percentage of bone volume per tissue volume (BV/TV; %), trabecular number (Tb.N; no./mm), trabecular separation (Tb.Sp.; μm), and trabecular thickness (Tb.Th; μm) were measured and analyzed using the SkyScan CT analyzer software. Three-dimensional reconstructed images of the sagittal and axial planes of the femoral metaphysis were generated using the SkyScan CTvox software (Skyscan).

Dual-energy X-Ray Absorptiometry (DEXA) and Imaging. Mouse whole body and femur bone mineral density (BMD; g/cm²) were analyzed using the Lunar PIXImus densitometer (GE Medical Systems, Madison, Wis.). Freshly euthanized WT and TUT7KO whole mice or femurs were placed on a specimen tray and scanned. For each specimen, the region of interest was selected and analyzed. Skeletal x-ray images of WT and TUT7KO were acquired using the IVIS Lumina XRMS Series III (PerkinElmer, Waltham, Mass.).

ELISAs. Plasma samples were collected from 8- and 16-weeks old WT and TUT7KO male and female mice (n≥5) by cardiac puncture. WT and TUT7KO plasma were analyzed by enzyme-linked immunosorbent assays (ELISA) to determine the levels of Osteocalcin (Biomedical Technologies, Stoughton, Mass.), CTX-I (MyBiosource, San Diego, Calif.), RANKL and OPG (R&D Systems) according to the manufacturer's instructions. To obtain the RANKL/OPG ratio for each animal, the level of RANKL was divided by the OPG level from the same animal and standardized based on WT levels.

Histology and Bone Histomorphometric Analysis. For histological analyses of osteoblasts and mineralized bone matrix, distal femurs from 8-weeks old male and female mice (n≥4) were dissected and fixed in 4% formaldehyde, dehydrated, and embedded undecalcified in methylmethacrylate resin. Sagittal sections were cut at 5 μm using a microtome and carbide knife. Sections were then stained with von Kossa and counterstained with 2% Toluidine Blue as previously described (Frara, et al. Journal of cellular physiology 231(1):72-83 (2016)). For histological analyses of osteoclasts, distal femurs from 16-weeks old male and female mice were fixed, decalcified, paraffin embedded, and cut into 5 μm sections. Sections were stained with Tartrate Resistant Acid Phosphatase (TRAP) and counterstained with 0.02% Fast Green in order to visualize osteoclasts on the bone surface. Bright field images were acquired using a Nikon Ti Eclipse inverted microscope (Nikon).

Quantitative histomorphometry was performed using the Osteomeasure software version 3.2.1 (Osteometrics, Decatur, Ga.). Images were acquired using a bright field microscope at 10× and 20× magnification equipped with a digital color video camera (Olympus, Center Valley, Pa.). Analyses were performed in an area 100-600 μm proximal to the growth plate. Three-dimensional parameters included trabecular number (Tb.N; No./mm) and trabecular separation (Tb.Sp; μm). Two-dimensional parameters included osteoblast number per bone perimeter (N.Ob/B.Pm; no./mm), percentage of osteoblast surface per bone surface (Ob.S/BS; %), osteoclast number per bone perimeter (N.Oc/B.Pm; no./mm), and percentage of osteoclast surface per bone surface (Oc.S/BS; %).

For dynamic histomorphometry, 8-weeks old WT and TUT7KO mice were injected subcutaneously (10 mg/kg) with Calcein AM 7 and 2 days before sacrifice. Femurs were collected, fixed, dehydrated, and embedded undecalcified in methylmethacrylate. Sections were cut, imaged and analyzed using a Nikon Eclipse Ti inverted microscope. Single-labeled surface, double layered surface, mineralizing surface, mineral apposition rate (MAR), and bone formation rate (BFR) were calculated as previously described (Abdelmagid, S M et al., The American journal of pathology, 184(3):697-713 (2014)).

Isolation and Analyses of Bone Marrow Derived and Calvaria Derived Osteoblast Cultures. Bone marrow progenitor cells from the long bones of 8-weeks old WT and TUT7KO mice were flushed and cultured in α-MEM containing 10% fetal bovine serum (FBS), 1% penicillin-streptomycin (PS), and 0.1% amphotericin-B (Amp-B). Calvarial primary osteoblasts from WT and TUT7KO five-day old pups were isolated and digested with 0.25% Trypsin and 0.1% Collagenase B.

For osteoblast differentiation, bone marrow progenitor cells or primary osteoblasts were cultured in 10% FBS, 10 mM β-glycerophosphate, 50 μg/mL ascorbic acid, and 10⁻⁷M dexamethasone. Osteoblast matrix maturation was assessed by alkaline phosphatase (ALP) staining and activity on undifferentiated (day 0) and differentiated (day 7) cultures using a kit (Sigma-Aldrich; Anaspec, Fremont, Calif.).

Osteoblast matrix mineralization was assessed by von Kossa staining. Briefly, bone marrow progenitor cells or primary osteoblasts were differentiated with osteogenic media for 21 days and fixed in 10% formalin. Fixed cultures were then stained with 5% silver nitrate and washed with dH₂O. Following washing, sodium carbonate and sodium thiosulfate were added to visualize mineralized nodules. Images for each well were analyzed using the NIS-Elements software.

Isolation and Analysis of Bone Marrow-Derived Osteoclast Cultures. Bone marrow progenitor cells from 8-weeks old WT and TUT7KO mice were obtained as previously described (Abdelmagid S M, et al., The Journal of biological chemistry 290(33):20128-20146 (2015)). Mature osteoclasts were analyzed by TRAP activity assay. Briefly, mature osteoclasts plated in 96-well plates were fixed with 10% formalin and washed with dH₂O. For TRAP activity assays, a 1:1 ratio of methanol:acetone was added to cultures followed by incubation with TRAP buffer (52 nM of Na+Tartrate in 0.1 M Na+-Acetate buffer) containing 0.1 mg/mL of p-nitrophenyl phosphate (p-NPP) (Thermo Fisher) for 1 hour at 37 ° C. Following incubation, 1 N NaOH was added to cultures and the optical density was read using a Synergy H4 microplate reader.

For TRAP staining, mature osteoclasts were incubated with TRAP buffer containing 1.5 mM Napthol-AS-MX phosphate and 0.5 mM Fast Red Violet. TRAP positive osteoclasts (n≥3) were counted and imaged using NIS-Elements software.

Osteoclast-mediated resorption was assessed by plating WT and TUT7KO bone marrow progenitor cells on Corning® OsteoAssay surfaces (Corning, Corning, N.Y.) and differentiated with M-CSF and RANKL as described above. Upon generation of mature osteoclasts, cultures were terminated using 10% bleach. Resorption areas were quantitated using NIS-Elements software.

Analysis of Osteoblast-Mediated Osteoclastogenesis using Co-Culture Assay. Primary calvarial osteoblasts from WT and TUT7KO were plated at 4.0×10⁴ cells/cm² in 48-well plates and treated with 1,25-dihydroxyvitamin D (10⁻⁸M) and prostaglandin E2 (PGE2; 10⁻⁶M) (Sigma-Aldrich). The next day, bone marrow non-adherent cells were co-cultured with osteoblasts at a density of 3.0×10⁵ cells/cm². Mature osteoclasts were evident within 7-10 days and were assessed by TRAP activity, staining, and count as described above.

Western Blotting. Cells were lysed in RIPA buffer and the total lysate protein was quantified using a BCA protein assay kit (Thermo), resolved by SDS-PAGE and transferred to a polyvinylidene fluoride (PVDF) membrane (BioRad, Hercules, Calif.). Membranes were probed overnight with primary antibodies against TUT7, Osterix, GAPDH, followed by incubation with the appropriate secondary HRP conjugated antibodies and the immunoreactive bands were visualized using chemiluminescent substrate (Millipore, Billerica, Mass.) and imaged on Syngene PXi system (Syngene, Rockville, Md.). Densitometric analysis was performed using the Syngene software.

Quantitative Real-Time PCR (RT-qPCR). Total RNA was isolated from WT and TUT7KO samples from tissue or cells as previously described (Sondag et al., Journal of cellular physiology 229(7):955-966 (2014)). Following RNA isolation, cDNA was prepared using a High Capacity cDNA Reverse Transcription kit (Life Technologies). Quantitative (q) RT-PCR was performed with the Step-one qPCR system with the 2× SYBR Green PCR Master Mix (Applied Biosystems, Foster City, Calif.) and relative mRNA expression of osteoclast-related genes was determined using the ▴▴C_{T} method with GAPDH as an internal control.

Osteogenesis Gene Expression Array. Total RNA was isolated from WT and TUT7KO osteoblasts using Qiazol and purified using an RNA extraction kit (Qiagen). Gene expression profiling was performed using a mouse osteogenesis RT² Profiler PCR array (Qiagen) and StepOnePlus system (Applied Biosystems). Relative gene expression was analyzed using the SABiosciences PCR array data analysis web portal.

Transfection and Luciferase Activity Assay. WT and TUT7KO osteoblasts or MC3T3-E1 osteoblast like cells were transfected using FuGene® HD transfection reagent (Promega). Cells were transfected or co-transfected with either PGL3 basic, PGL3-OSX, pcDNA3, pcDNA3-FLAG-TUT7 using FuGene® HD transfection reagent. Forty-eight hour post-transfection, cells were harvested and assayed using the Dual Luciferase assay system (Promega). The luciferase activity values were normalized based on Renilla values to correct for variation in transfection efficiency.

Data and Statistical Analyses. For all data generated, differences between individual groups were analyzed using Prism software version 5.04 (GraphPad, La Jolla, Calif.). All experiments were repeated 3-5 times with similar results. In cases involving the comparison of two groups, an unpaired t-test was performed. In cases when multiple groups were being compared, a one-way analysis of variance (1-way ANOVA) was employed along with Tukey's multiple comparison post hoc test. Group means or means±standard error of the mean (±SEM) was graphed. All differences where p<0.05 were regarded as statistically significant.

### Results

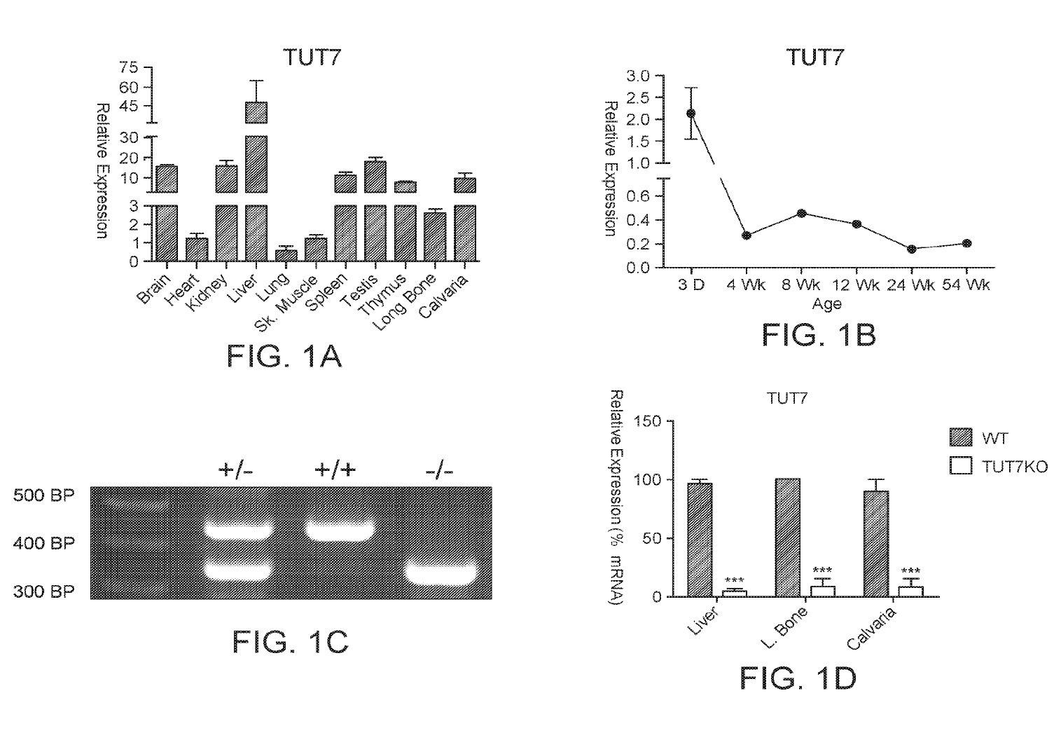

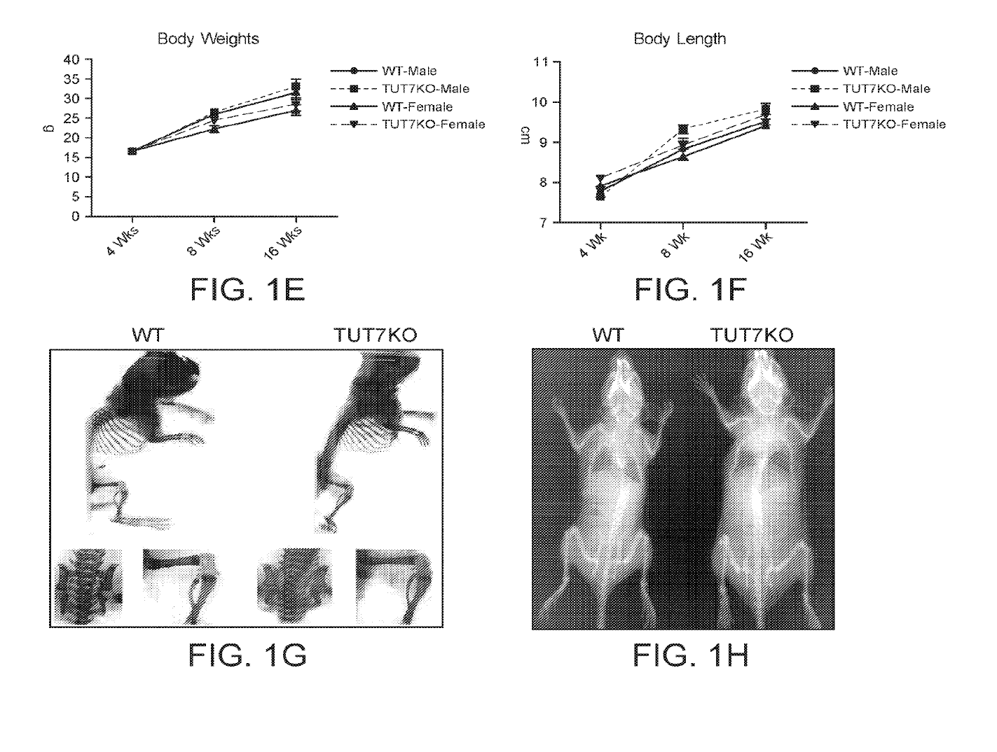

TUT7 is differentially expressed with age. We first quantified the expression of TUT7 in bone and soft tissues of mice by qPCR. Our data showed that TUT7 mRNA was highly expressed in liver, brain and kidney of 8-weeks old mice (FIG. 1A). Interestingly, TUT7 mRNA was also highly expressed in calvaria and long bones. Next, we determined the expression of TUT7 in aging bone and discovered that the expression of TUT7 in bone was highest at day 3 but was drastically reduced by 4 weeks of age and then remained low throughout ontogeny (FIG. 1B). These findings indicated that TUT7 may play an important role in early postnatal development of the skeletal system.

TUT7 deficient mice develop normally. To determine the role of TUT7 in skeletal development and remodeling we derived a line of TUT7/Zcchc6 gene mutant mice and used PCR to distinguish homozygous (KO, −/−), heterozygous (Het, +/−), and wildtype (WT, +/+) genotypes (FIG. 1C). In addition, we confirmed the absence of TUT7 mRNA expression in multiple tissues of the KO mice by qPCR (FIG. 1D). TUT7KO mice developed normally and absence of the gene did not have a significant effect on either body weight (FIG. 1E) or body length (FIG. 1F). Furthermore, there did not seem to be any dramatic or noticeable differences in morphology or skeletal structure during early development (FIG. 1G) or adult stage (FIG. 1H).

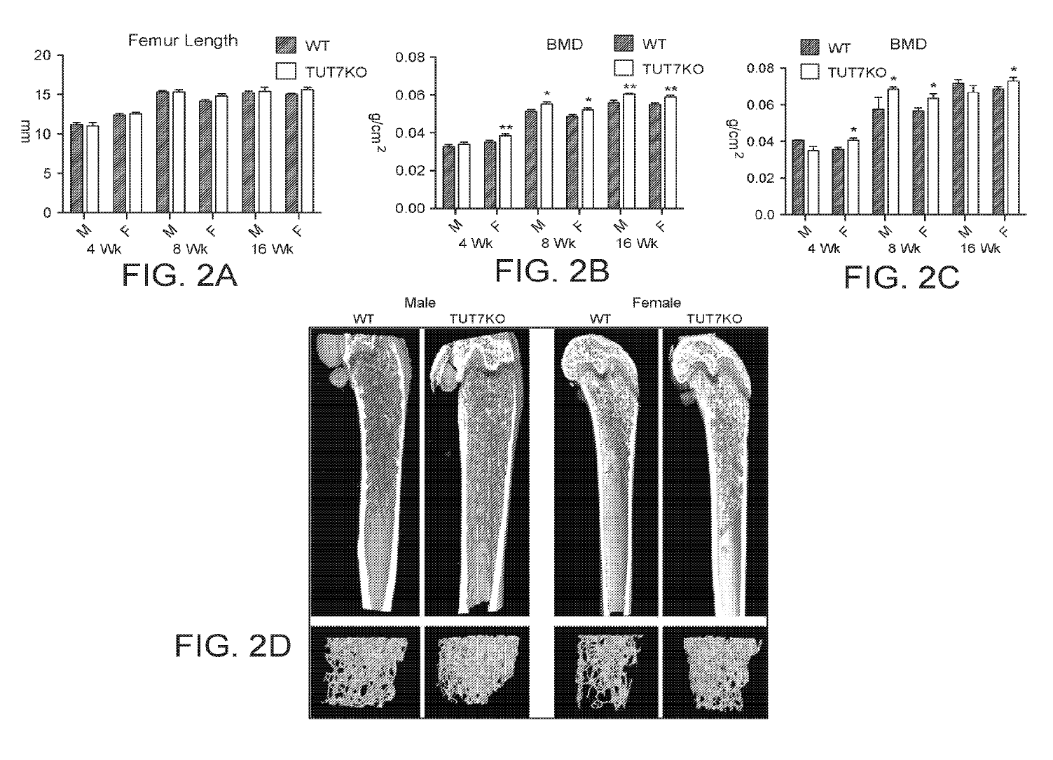

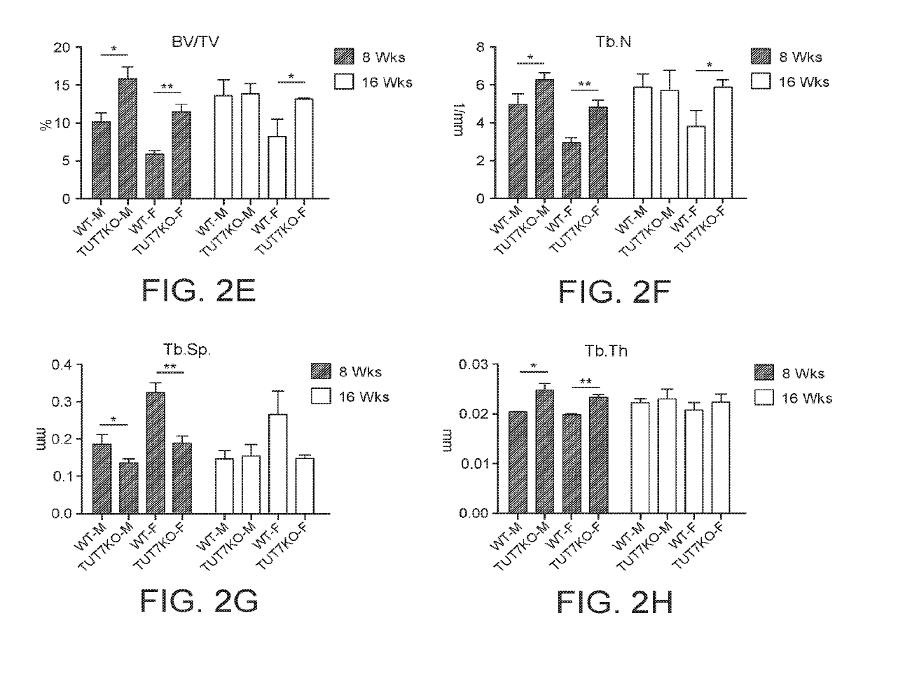

Absence of TUT7 enhances bone mass in vivo. Analyses of the bones of TUT7KO mice showed no difference in femur length but TUT7KO mice had increased whole body (FIG. 2B) and femoral BMD compared to WT mice (FIG. 2C). Interestingly, TUT7KO mice femurs showed a dramatic increase in the trabecular bone mass compared to WT mice (FIG. 2D). Further analyses revealed a significant increase in BV/TV, Tb.N, Tb. Sp., and Tb.Th in 8 weeks old TUT7KO mice compared to WT mice (FIGS. 2E-H). Interestingly, the increase in bone mass was greater in TUT7KO female mice than in males. Furthermore, microCT analysis of femurs from 16-weeks old mice revealed a significant increase in BV/TV and Tb.N in TUT7KO female mice but not in male mice (FIGS. 2E-H). This indicated that TUT7 may have an age-dependent and gender-specific role in the regulation of bone mass, for reasons not clear at present.

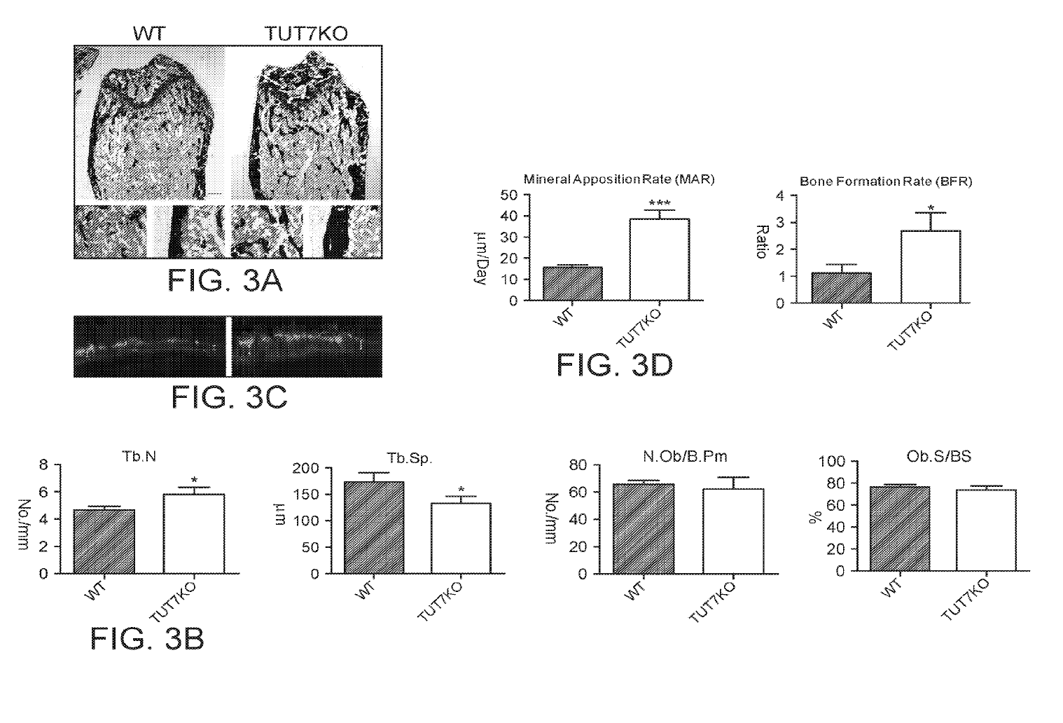

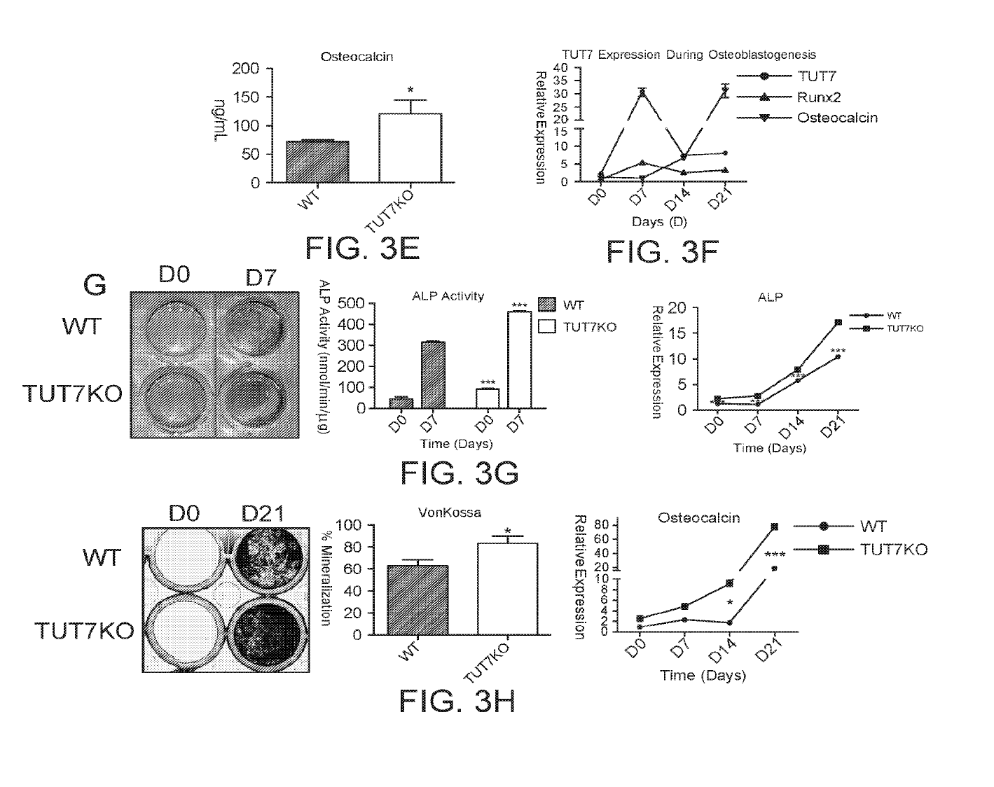

Deficiency of TUT7 enhances bone formation in vivo and increases osteoblast differentiation ex vivo. To understand the cause of increase in bone mass in TUT7KO mice, we examined bone formation in vivo by histomorphometric analyses. Femurs from 8-weeks old WT and TUT7KO mice were stained for von Kossa and analyzed. Femurs from TUT7KO mice showed a significant increase in trabecular number (Tb.N) and trabecular spacing (Tb.Sp.) (FIGS. 3A-B). Interestingly, there seemed to be no significant difference in the number of osteoblasts (N.Ob/B.Pm) or osteoblast bone surface (Ob.S/BS) in these mice. This indicated that the increase in bone matrix mineralization may be due to an increased activity of the osteoblasts rather than an increase in numbers in the TUT7KO mice. To test this, we injected WT and TUT7KO mice with Calcein to assess the dynamic mineralization rate in vivo. Our results showed that TUT7KO mice had ˜150% increase in mineral apposition rate (MAR) and bone formation rate (BFR) compared to WT mice (FIGS. 3C-D). Furthermore, TUT7KO mice showed a significant increase (p<0.05) in the serum levels of osteoblast marker osteocalcin compared to the levels in wild type mice (FIG. 3E). These results demonstrated that absence of TUT7 enhances bone formation and mineralization in vivo.

Next, we examined the TUT7 expression during osteoblastogenesis, and determined its role in osteoblast differentiation. Interestingly, TUT7 expression was greatly enhanced at day 7 of osteoblast differentiation, but decreased thereafter (FIG. 3F). Osteoblasts derived from TUT7KO mice did not expressed TUT7 but showed a significant increase in ALP staining, activity, and mRNA expression compared to osteoblasts derived from WT mice (FIG. 3G). Expression of ALP was significantly high in TUT7KO cultures compared to WT even without osteogenic media treatment (D0) indicating that endogenously, TUT7 may regulate specific factors related to osteoblast commitment. Next, we assessed the role of TUT7 in late stage osteoblast differentiation and matrix mineralization. Osteoblast cultures from TUT7KO mice showed a significant increase in osteoblast matrix mineralization and osteocalcin expression compared to cultures derived from WT mice (FIG. 3H). These results demonstrated that absence of TUT7 has a significant impact on osteoblast differentiation and matrix mineralization in vivo.

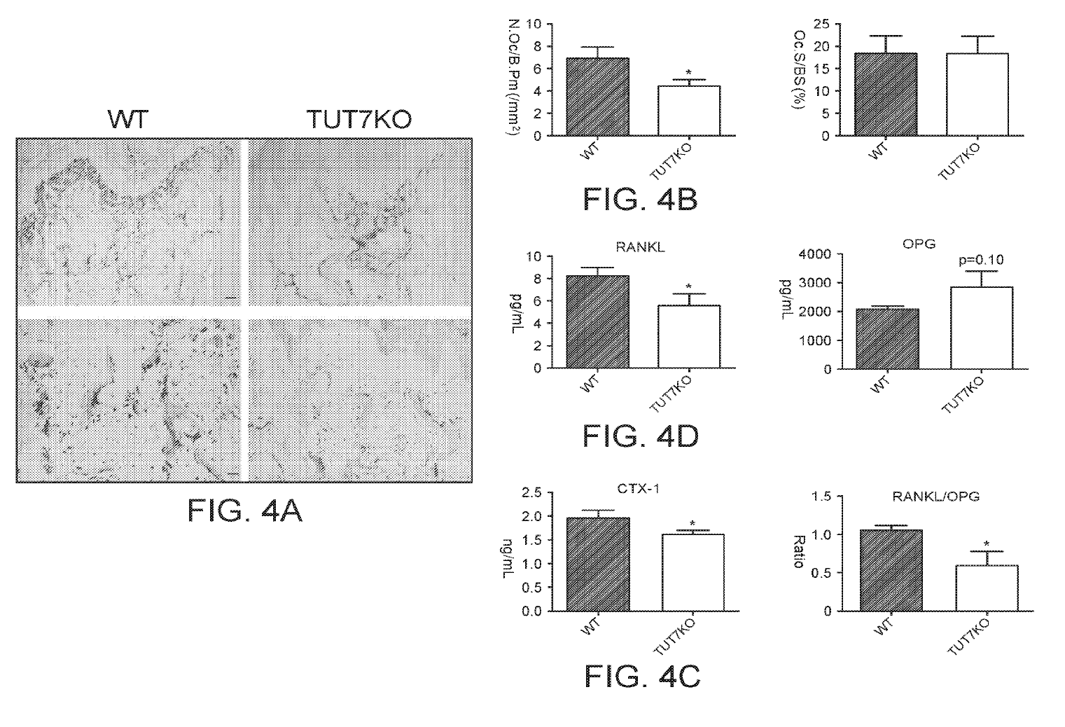

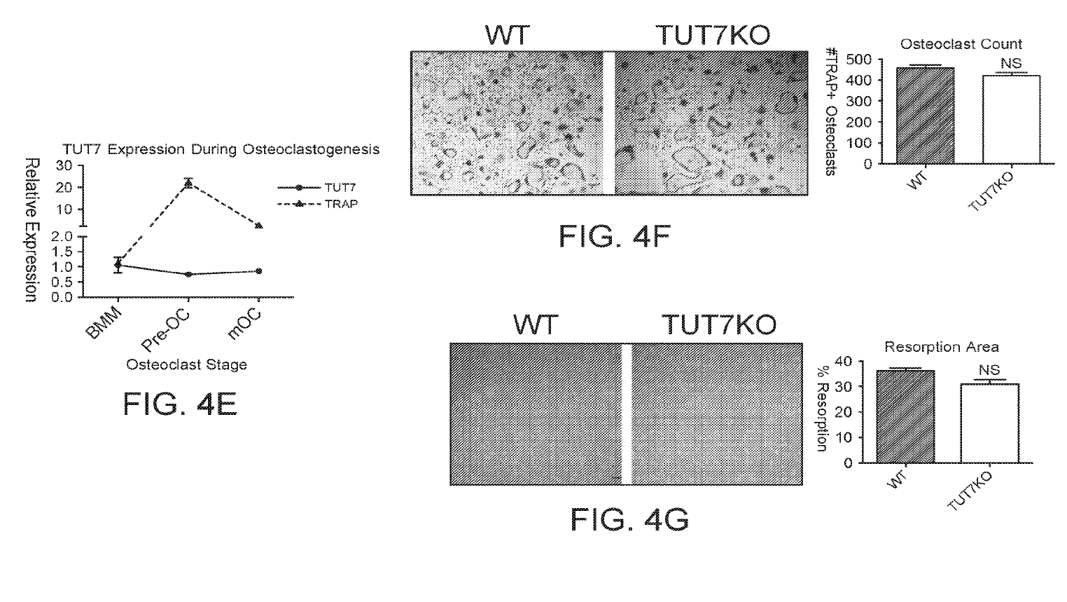

Absence of TUT7 inhibits osteoclasts in vivo, but not ex vivo. Next, we evaluated the role of TUT7 in osteoclastogenesis to determine if this may contribute to the enhanced bone mass observed in the TUT7KO mice. TRAP staining and histomorphometric analyses in the TUT7KO mice revealed a significant reduction in the number of osteoclasts (N.Oc/B.Pm), but no difference in the osteoclast surface (Oc.S/BS) compared to WT mice (FIG. 4A-B). This indicated that the absence of TUT7 affect the number of mature osteoclasts but not their size or morphology in vivo. Serum levels of CTX-1 and RANKL were significantly reduced in the TUT7KO mice but no significant difference in the levels of OPG was detected; however, there was a reduction in the overall RANKL/OPG ratio in the TUT7KO mice compared to WT mice (FIGS. 4C-D). These results indicated that TUT7 may play a role in the differentiation and activity of osteoclasts in vivo.

Since the absence of TUT7 suppressed the generation of mature osteoclasts in vivo, we evaluated the role of TUT7 during osteoclastogenesis ex vivo. Expression of TUT7 mRNA during osteoclastogenesis in vitro was not altered during osteoclast differentiation from WT mice (FIG. 4E). Additionally, when bone marrow progenitor cells from WT and TUT7KO mice were differentiated towards osteoclasts, there was no significant difference in osteoclast differentiation as determined by TRAP staining and count or osteoclast resorption (FIGS. 4F-G). Taken together our data indicated that the absence of TUT7 affected osteoclast differentiation and function in vivo but not ex vivo suggesting the involvement of other factors in osteoclast differentiation and function in vivo that may be regulated by TUT7.

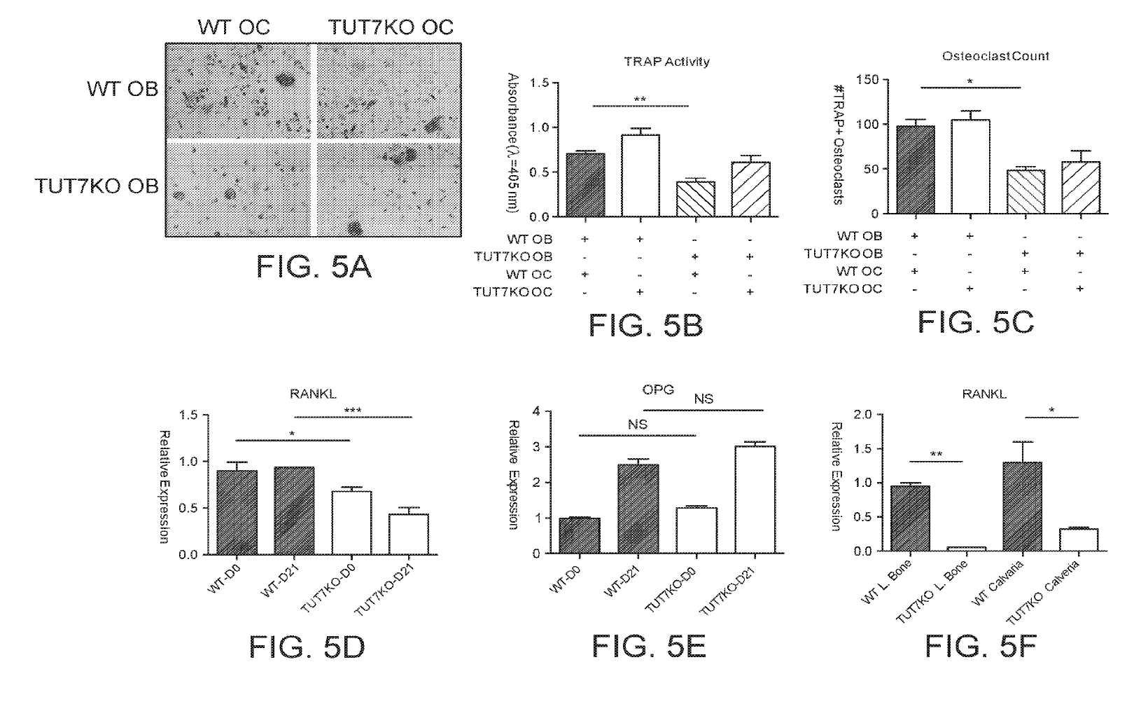

Absence of TUT7 regulates osteoblast-mediated osteoclastogenesis. To address the discrepancy observed in TUT7KO osteoclasts in vivo and ex vivo, we used an osteoblast and osteoclast “mix and match” co-culture system. There was no difference in osteoclast differentiation in WT osteoblasts (OB) co-cultured with WT osteoclasts (OC) compared to WT OB co-cultured with TUT7KO OCs. Importantly, there was a significant reduction in osteoclast differentiation when TUT7KO OBs were co-cultured with WT OCs compared to WT OBs co-cultured with WT OCs as shown by TRAP staining, activity, and count (FIGS. 5A-C). Furthermore, the expression of RANKL, but not OPG, was significantly downregulated in TUT7KO osteoblasts compared to WT osteoblasts (FIGS. 5D-E) and also in long bones and calvaria of TUT7KO mice compared to WT mice (FIG. 5F). These results indicated that the role of TUT7 in osteoclast differentiation is indirectly mediated through regulation of RANKL expression in osteoblasts.

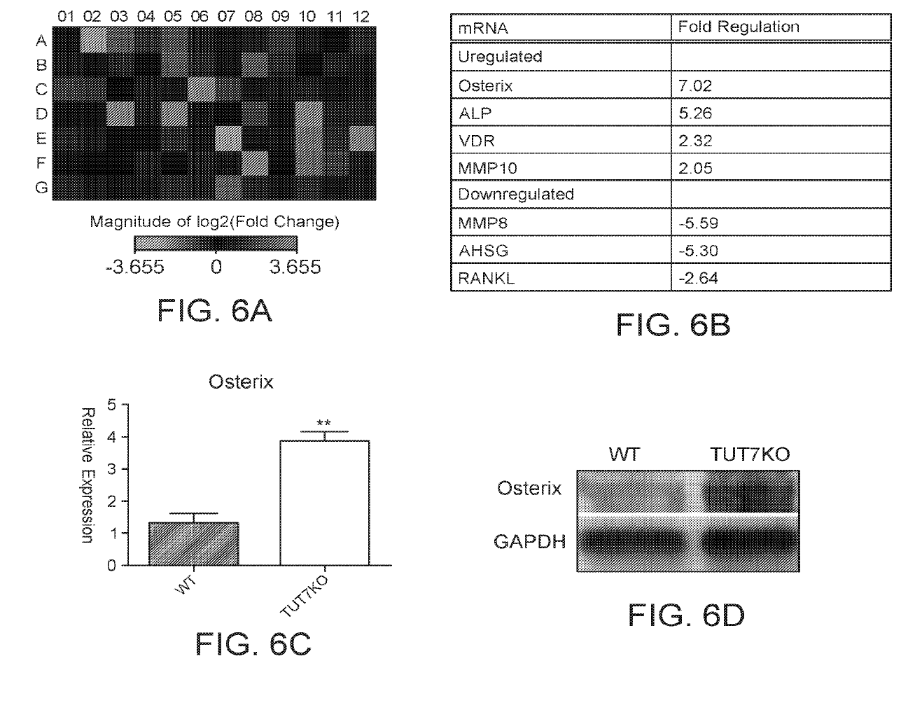

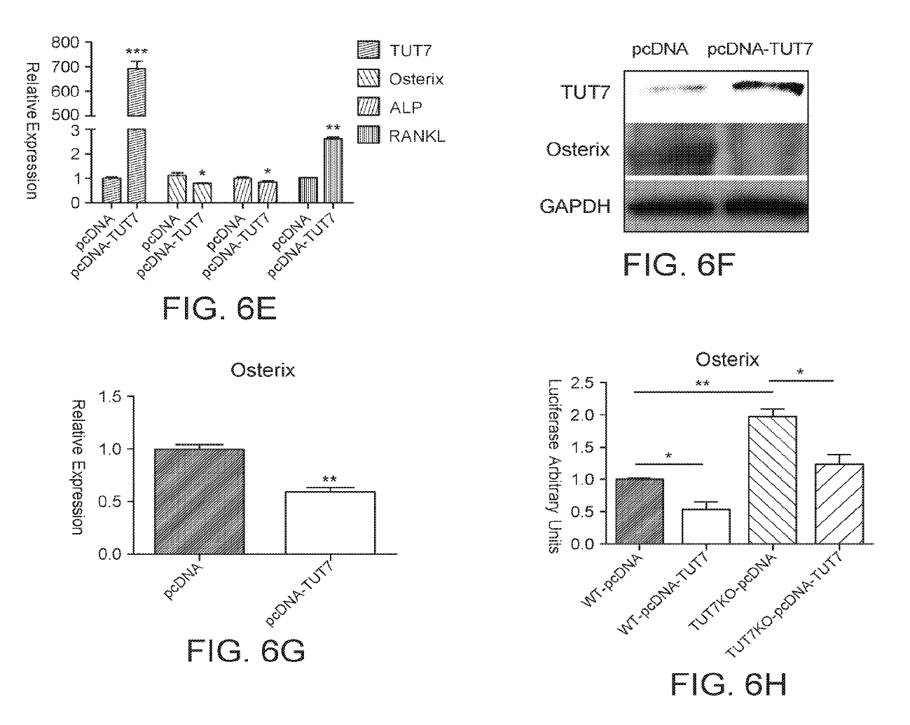

TUT7 regulates the expression of the master transcription factor Osterix in osteoblasts. We also analyzed osteoblast-related genes expression using a PCR-based osteogenesis gene array and identified a number of dysregulated genes including a seven-fold increase in the expression of the transcription factor Osterix mRNA (FIGS. 6A-B). The upregulation of Osterix expression was confirmed at both the mRNA and protein levels (FIGS. 6C-D) indicating that TUT7 is a negative regulator of Osterix expression. Importantly we found that overexpression of TUT7 decreased the expression of Osterix and ALP mRNAs, and increased RANKL mRNA expression (FIG. 6E). Furthermore, MC3T3-E1 osteoblast-like cells overexpressing TUT7 gene, had significantly reduced levels of Osterix protein and transcriptional activity (FIGS. 6F-G). We also overexpressed TUT7 gene in WT and TUT7KO osteoblasts and examined the transcriptional activity of Osterix by luciferase activity assay (FIG. 6H). Significantly high levels of Osterix transcriptional activity was observed in the TUT7KO osteoblasts but was reduced in WT cells overexpressing TUT7 as expected. Interestingly, when TUT7 was ectopically expressed in osteoblasts derived from TUT7KO mice, Osterix activity was restored to WT control levels. Overall, this data demonstrated that TUT7 negatively regulates Osterix expression and activity in osteoblasts.

### Example 2

Interleukin-1β (IL-1β) is the major cytokine involved in cartilage catabolism in osteoarthritis (OA) and induces the expression of pro-inflammatory cytokine IL-6. IL-6 is known to induce the expression of MMP-13 and inhibit type-II collagen expression. Cytoplasmic RNA nucleotidyl transferases catalyze the addition of nucleotides to the 3′ end of mRNAs. However, the expression or role of RNA nucleotidyl transferases in regulating cytokine expression in OA is unknown. The aim of this study was to investigate whether RNA nucleotidyl transferase ZCCHC6, a recently identified member of the ribonucleotidyl transferases superfamily, is expressed in OA cartilage, identify the cytokines regulated by ZCCHC6 in chondrocytes, and whether ZCCHC6 is involved in the regulation of IL-6 expression in OA chondrocytes.

Chondrocytes were derived by enzymatic digestion of human cartilage obtained from OA patients (n=14) undergoing knee joint replacement. Chondrocytes were stimulated with IL-1β (5 ng/ml) or treated with Actinomycin D (5 μg/ml) or NFκB inhibitor SC514 (75 μM) or JNK inhibitor (25 μM). Total RNA from grounded cartilage and from chondrocytes was purified using Qiagen RNeasy kit (Qiagen). Reverse transcription was performed using the Quantitect Reverse Transcription kit and the ZCCHC6 or IL-6 mRNA was quantified using TaqMan assays. SiRNA-mediated depletion of ZCCHC6 in human chondrocytes was used to study the effect on inflammatory cytokine expression using a cytokine array (Ray Biotech). Protein expression of IL-6 was studied using Western immunoblotting and quantified by ELISA in culture supernatants. Results were derived using Sigma Plot 12.3 package and p<0.05 was considered significant.

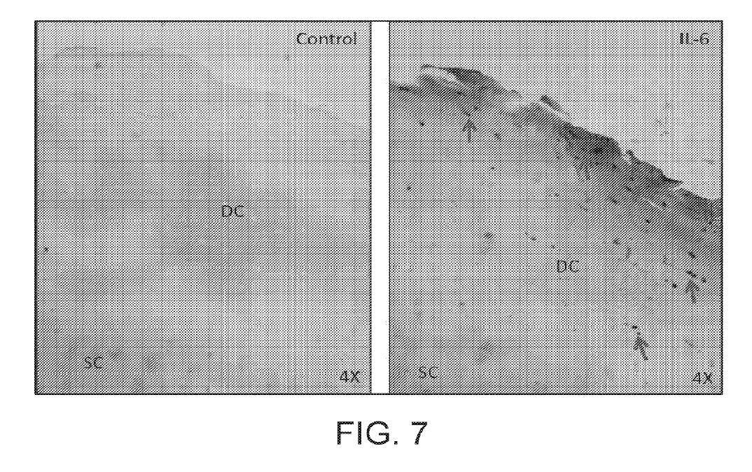

The results showed higher expression of nucleotidyl transferase ZCCHC6 in the damage cartilage compared to unaffected cartilage. See FIG. 7. Higher expression of IL-6 (6.0 fold±1.44) in damaged cartilage compared to smooth cartilage (n=3; p<0.05) from OA patients was also observed. We further demonstrate that IL-1β stimulation resulted in a significant increase in the expression of ZCCHC6 (11.3-fold±1.6) and the mRNA of the inflammatory cytokine IL-6 (4956-fold±40.6) in human chondrocytes (n=4; p<0.05). Similar increase in the protein expression of both ZCCHC6 and IL-6 was also observed. Depletion of ZCCHC6 significantly decreased the expression of IL-6 mRNA (˜77-95%) and protein in IL-1β-stimulated chondrocytes and IL-6 mRNAs in IL-1β-stimulated chondrocytes treated with ZCCHC6 siRNA had shorter poly-A tails (n=3; p<0.05). Cell supernatants from control or ZCCHC6 siRNA treated chondrocytes stimulated with IL-1β were analyzed using a cytokine array. A subset of cytokines including IL-6 was substantially decreased by loss of ZCCHC6. Our results also showed that the IL-1β-induced activation of NF-κB has no role in regulation of ZCCHC6 expression in OA chondrocytes and ZCCHC6 expression was regulated by JNK-MAPKs (n=3; p<0.05). Additionally, OA chondrocytes transfected with ZCCHC6 siRNA also showed a decrease (˜86%) in constitutive IL-6 mRNA expression (n=3; p<0.05).

Taken together, the results demonstrate for the first time that ZCCHC6 is highly expressed in damaged human cartilage compared from OA patients. Furthermore, ZCCHC6 modulates IL-6 expression in human chondrocytes at the post-transcriptional level by influencing cytokine mRNA stability.

### Example 3

Interleukin-1β stimulates the expression of several inflammatory mediators including IL-6 which play an important role in the pathogenesis of osteoarthritis. Interleukin-6 is a pro-inflammatory cytokine that activates the transcription of its target genes via formation of an IL-6 receptor complex involving a membrane bound IL-6 receptor (IL-6R), soluble IL-6R (sIL-6R) and gp130 followed by activation of STAT1/STAT3 pathway.

In our preliminary studies (Example 2 above) employing standard immunohistochemical techniques, we found that chondrocytes present in damaged cartilage express IL-6 while little or no IL-6 expressing cells were found in the cartilage area that stained positive with Safranin-O. Thus, taken together there exists a strong correlation between the expression of IL-6 and cartilage degradation in human OA patients and in animal models of OA. Recently, a novel post-transcriptional regulation of cytokine expression mediated by the 3′ end modifications of miRNAs that target IL-6 mRNA by a member of TUTase family of enzymes has been shown in cancer but post-transcriptional regulation of IL-6 expression and expression and function of TUTases in OA has not been explored in detail. In our preliminary studies, we identify a novel TUTase ZCCHC6 that is differentially expressed in human OA cartilage and chondrocytes and its expression is modulated by IL-1β. We therefore propose to investigate whether ZCCHC6 post-transcriptionally regulates IL-6 expression in human chondrocytes stimulated with IL-1β and we will determine the impact of zcchc6 deletion on IL-6 expression and disease severity using a zcchc6 knock-out mouse.

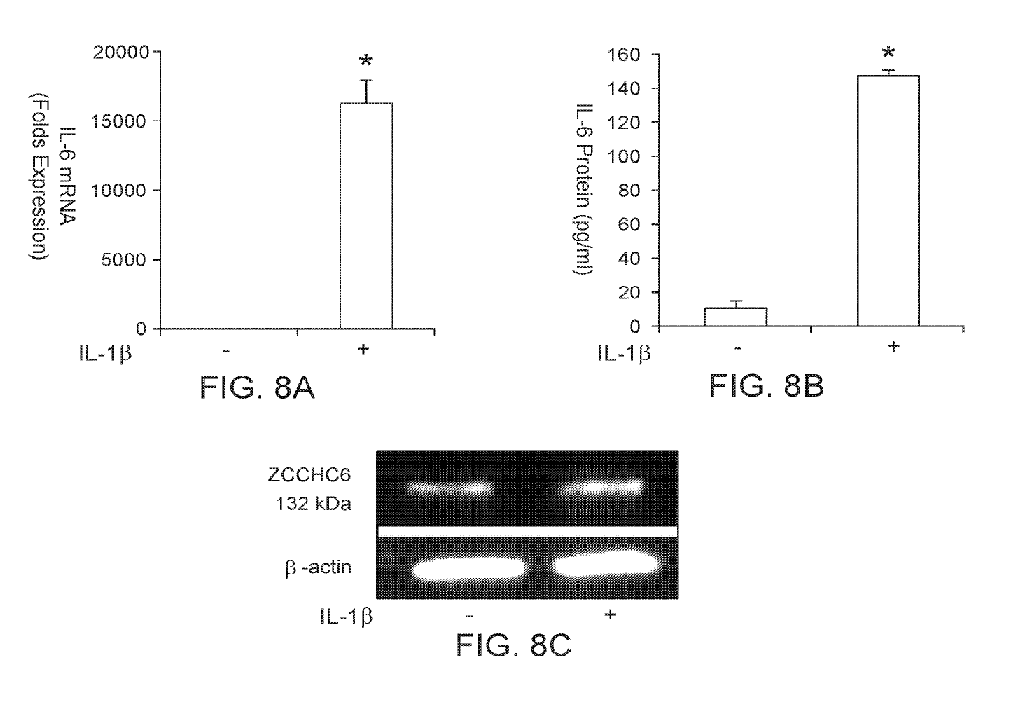

IL-1β-induces expression and production of IL-6 and ZCCHC6 by human OA chondrocytes in vitro. We next determined whether IL-1β induces expression of IL-6 correlates with the expression of ZCCHC6 in human OA chondrocytes. Human OA chondrocytes were cultured and treated with IL-1β as above and the mRNA expression of IL-6 was quantified by TaqMan assay and secreted IL-6 was quantified using an ELISA kit specific for human IL-6 (R&D Systems, Catalog No. D0650, sensitivity of 2.17 pg IL-6/ml). ZCCHC6 protein expression was determined by Western immunoblotting using an antibody specific for the N-terminus of the protein (Santa Cruz, sc-137947). These results, shown in FIG. 8A, demonstrate that in untreated chondrocytes expression of IL-6 mRNA was barely detectable and negligible amounts of IL-6 were secreted into the medium. In contrast, stimulation of human chondrocytes with IL-1β resulted in high levels of expression of IL-6 mRNA and these cells also secreted large amounts of IL-6 in the culture medium (FIG. 8B). Also, in chondrocytes stimulated with IL-1β, expression of ZCCHC6 protein was highly induced (FIG. 8C). These data indicate that IL-1β is a potent inducer of both IL-6 and ZCCHC6 mRNA and protein expression in human OA chondrocytes. Furthermore, OA chondrocytes that showed high levels of ZCCHC6 expression also produced more IL-6 in the culture supernatant.

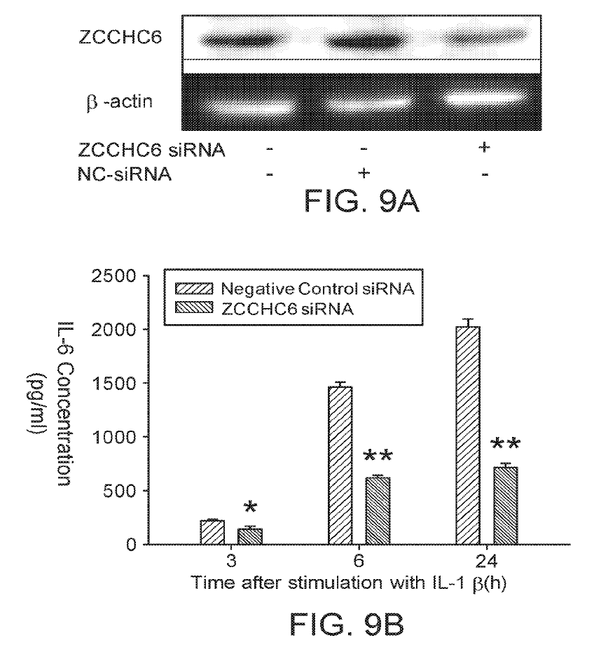

siRNA-mediated knockdown of ZCCHC6 in human chondrocytes inhibited IL-6 expression. To determine if ZCCHC6 plays a direct role in IL-6 expression in OA chondrocytes, human OA chondrocytes were transfected with 100 nanomoles of ZCCHC6 specific targeting siRNAs (OnTarget Plust, SMARTPOOL siRNAs, Dharmacon) using the Amaxa system and effective knockdown relative to control, non-targeting siRNA was confirmed by immunoblotting using a ZCCHC6-specific antibody (Santa Cruz Biotechnology, sc-137947). Viability of transfected chondrocytes was determined by Trypan Blue exclusion assay and no significant effect on chondrocyte viability was observed. Transfection of human OA chondrocytes with the ZCCHC6 targeting SMARTPOOL siRNAs knocked down the ZCCHC6 protein expression by >75% in the transfected OA chondrocytes (FIG. 9A). To determine the role of ZCCHC6 in IL-6 expression, human OA chondrocytes with knocked down ZCCHC6 expression were stimulated with IL-1β and the secreted IL-6 protein in the culture supernatants was quantified using a sandwich ELISA assay kit (R&D Systems Cat #D6050). Compared to the levels detected in culture supernatants of chondrocytes transfected with non-targeting siRNAs, knockdown of ZCCHC6 resulted in a dramatic decrease in IL-6 protein secretion in culture supernatants of human OA chondrocytes transfected with ZCCHC6 targeting siRNAs (FIG. 9B). Importantly, siRNA-mediated knock down of ZCCHC11 expression had no effect on IL-6 protein expression in human chondrocytes. Taken together, these data indicate a correlation between the expression of ZCCHC6 and the expression of IL-6 in human OA chondrocytes.

From the above description, those skilled in the art will perceive improvements, changes and modifications. Such improvements, changes, and modifications are within the skill of those in the art and are intended to be covered by the appended claims. All patents, patent applications, and publication cited herein are incorporated by reference in their entirety.

The following is claimed:

1. A composition for preventing bone loss and/or stimulating bone healing in a subject in need thereof, the composition comprising:
a pharmaceutically acceptable carrier; and
a therapeutically effective amount of a zinc-finger CCHC domain-containing protein 6 (ZCCHC6) inhibitor.

2. The composition of claim 1, wherein the subject has a bone fracture.

3. The composition of claim 1, wherein the subject is human.

4. The composition of claim 1, wherein the ZCCHC6 inhibitor is an anti-ZCCHC6 antibody or an active fragment thereof.

5. The composition of claim 4, wherein the antibody is a monoclonal antibody.

6. The composition of claim 4, wherein the antibody is a humanized antibody.

7. The composition of claim 4, wherein the antibody is a human ZCCHC6 antibody.

8. The composition of claim 1, wherein the ZCCHC6 inhibitor is siRNA.

9. The composition of claim 8, wherein the siRNA is human ZCCHC6 siRNA.

10. The composition of claim 1, wherein the pharmaceutically acceptable carrier is a biocompatible material.

11. The composition of claim 10, wherein the biocompatible material is a collagen sponge.

12. The composition of claim 1, wherein the pharmaceutically acceptable carrier is a polymer.

13. The composition of claim 1, wherein the pharmaceutically acceptable carrier is a nanoparticle.

14. The composition of claim 1, further comprising mesenchymal stem cells (MSCs).

15. A method of preventing bone loss and/or stimulating bone healing in a subject in need thereof, comprising administering a therapeutically effective amount of a zinc-finger CCHC domain-containing protein 6 (ZCCHC6) inhibitor to the subject.

16. The method of claim 15, wherein the subject has a bone fracture.

17. The method of claim 15, wherein the subject is human.

18. The method of claim 15, wherein the ZCCHC6 inhibitor is an anti-ZCCHC6 antibody or an active fragment thereof.

19. The method of claim 18, wherein the antibody is a monoclonal antibody.

20. The method of claim 18, wherein the antibody is a humanized antibody.

21. The method of claim 18, wherein the antibody is a human ZCCHC6 antibody.

22. The method of claim 15, wherein the ZCCHC6 inhibitor is siRNA.

23. The method of claim 22, wherein the siRNA is human ZCCHC6 siRNA

24. The method of claim 15, wherein MSCs are also administered to the subject.

25. The method of claim 15, wherein the ZCCHC6 inhibitor is administered using a pharmaceutically acceptable carrier.

26. The method of claim 15, wherein the pharmaceutically acceptable carrier is a biocompatible material.

27. The method of claim 26, wherein the biocompatible material is a collagen sponge.

28. The method of claim 25, wherein the pharmaceutically acceptable carrier is a polymer.

29. The method of claim 25, wherein the pharmaceutically acceptable carrier is a nanoparticle.
